(19) United States
(12) Patent Application Publication
(10) Pub. No.: US 2025/0049794 A1
(43) Pub. Date: Feb. 13, 2025

(54) PHARMACEUTICAL FORMULATIONS OF GPR119 AGONISTS AND USES THEREOF

(71) Applicant: MANKIND PHARMA LTD., New Delhi (IN)

(72) Inventors: Dheeraj FULARA, Gurugram (IN); KS HAREEN, Gurugram (IN); Rashmi Ranjan PANDA, Gurugram (IN); Chithambaram MUTHULINGAM, Gurugram (IN); Santosh Kumar RAI, Gurugram (IN); Anil KUMAR, Gurugram (IN)

(21) Appl. No.: 18/799,836

(22) Filed: Aug. 9, 2024

Related U.S. Application Data
(63) Continuation of application No. PCT/IB2024/057713, filed on Aug. 8, 2024.

(30) Foreign Application Priority Data
Aug. 11, 2023 (IN) 202311054015

(51) Int. Cl. A61K 31/506; A61K 9/10; A61K 9/20; A61K 31/454; A61K 31/4545; A61K 47/40

(52) U.S. Cl. CPC A61K 31/506; A61K 9/10; A61K 9/2009; A61K 9/2018; A61K 9/2027; A61K 9/204; A61K 9/2054; A61K 31/454; A61K 31/4545; A61K 47/40

(57) ABSTRACT

The present disclosure relates to oral pharmaceutical formulations comprising a compound of Formula I or a pharmaceutically acceptable salt thereof

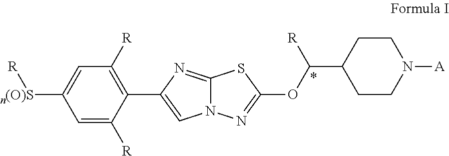

wherein R, A and n are as described herein. The present disclosure also relates to processes for their preparation and their use in treating disorders such as cardiovascular and metabolic disorders, including diabetes.

This application is a continuation of International Application No. PCT/IB2024/057713, filed Aug. 8, 2024, which claims the benefit of Indian Patent Application number 202311054015, filed on Aug. 11, 2023, each of which is hereby incorporated by reference.

### FIELD OF THE INVENTION

The present invention relates to oral pharmaceutical formulations comprising a compound of Formula I or a pharmaceutically acceptable salt thereof

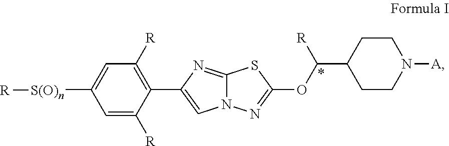

wherein R, A and n are as described herein. The present invention also relates to processes for their preparation and their use in treating disorders such as cardiovascular and metabolic disorders, including diabetes.

### BACKGROUND OF THE INVENTION

Approximately 415 million people live with diabetes worldwide, and an estimated 193 million people have undiagnosed diabetes. Type-2 diabetes accounts for more than 90% of patients with diabetes and leads to microvascular and macrovascular complications that cause profound psychological and physical distress to both patients and carers and put a huge burden on health-care systems. Type-2 diabetes mellitus is a chronic metabolic condition characterised by chronic hyperglycemia due to deficiency in the secretion and/or function of insulin (i.e. insulin resistance). Type-2 diabetes mellitus is known to be associated with a reduced life expectancy and increased morbidity. Although cardiovascular disease is the commonest cause of mortality and morbidity, Type-2 diabetes mellitus is also associated with an increased risk of other diseases including cancer, chronic liver disease including non-alcoholic fatty liver disease (NAFLD) such as non-alcoholic fatty liver (NAFL), non-alcoholic steatohepatitis (NASH), dyslipidaemias, and related disorders, cognitive decline and accelerated arthritis, in addition to the microvascular and macrovascular complications.

After long duration of disease, most patients with Type-2 diabetes will eventually fail on oral therapy and become insulin dependent with the necessity for daily injections and multiple daily glucose measurements. Oral antidiabetic drugs conventionally used in therapy (such as e.g. first- or second-line, and/or mono- or (initial or add-on) combination therapy) include, for example, metformin, sulphonylureas, thiazolidinones, glinides, and α-glucosidase inhibitors.

The high incidence of therapeutic failure is a major contributor to the high rate of long-term hyperglycaemia-associated complications or chronic damages (including micro- and macrovascular complications such as e.g. diabetic nephropathy, retinopathy or neuropathy, or cardiovascular complications) in patients with Type-2 diabetes.

U.S. Pat. No. 10,208,030 describes GPR119 agonists and processes for their preparation.

There is still a need to provide new oral pharmaceutical formulations that may be used to treat disorders such as diabetes. The present invention addresses this need.

### SUMMARY OF THE INVENTION

In one aspect, the present invention relates to an oral pharmaceutical composition comprising a therapeutically effective amount of a compound of Formula I

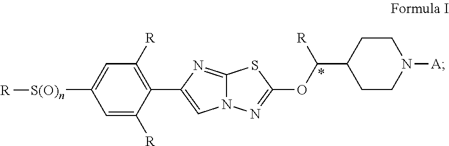

or a pharmaceutically acceptable salt thereof, wherein each R is independently selected from H, halogen, substituted or unsubstituted C₁₋₄ alkyl, and substituted or unsubstituted C₁₋₄ alkoxy;

the variable A is selected from hydrogen, substituted or unsubstituted C₁₋₄ alkyl, substituted or unsubstituted phenyl, substituted or unsubstituted (C₁₋₄ alkyl)phenyl, substituted or unsubstituted 5-6 membered heteroaryl containing one or more heteroatoms selected from N, O, and S, substituted or unsubstituted heterocyclyl containing one or more heteroatom selected from N, O, and S, and substituted or unsubstituted C₁₋₄ alkoxycarbonyl; wherein the substituents are selected from halogen, substituted or unsubstituted C₁₋₄ alkyl, and substituted or unsubstituted C₁₋₄ alkoxy;
n is 0, 1, 2, or 3; and
* denotes a stereocenter;
   and one or more pharmaceutically acceptable excipients,
   wherein optionally the compound of Formula I has a particle size D₉₀ of less than about 200 μm.

In one embodiment of any of the compositions described herein, the compound of Formula I is a compound of Formula IA, IB, IC, ID or IE:

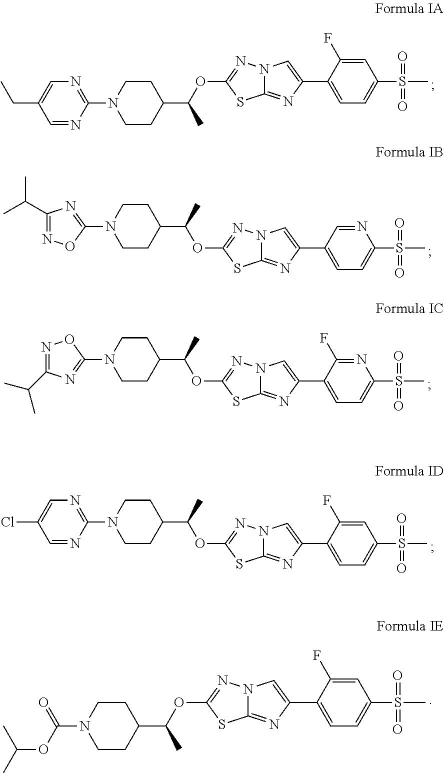

In one embodiment of any of the compositions described herein, the compound of Formula I is a compound of Formula IA (2-((S)-1-(1-(5-ethylpyrimidin-2-yl) piperidin-4-yl) ethoxy)-6-(2-fluoro-4-(methylsulfonyl)phenyl) imidazo [2,1-b][1,3,4]thiadiazole):

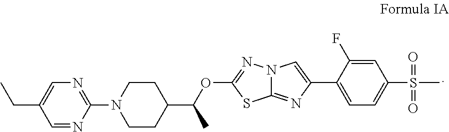 [SMILES: CCc1cnc(N2CCC([C@H](C)Oc3nn4cc(-c5ccc(S(C)(=O)=O)cc5F)nc4s3)CC2)nc1]

In one embodiment of any of the compositions described herein, the composition includes a therapeutically effective amount of a compound of Formula I.

In one embodiment of any of the compositions described herein, the one or more pharmaceutically acceptable excipients are selected from a diluent, a disintegrant, a binder, a lubricant, a glidant, a pH modifier, a surfactant, a solubilizer, an anti-oxidant, or any combination of any of the foregoing.

In one embodiment of any of the compositions described herein, the compound of Formula I, such as a compound of Formula IA, IB, IC, ID or IE, has a particle size D₉₀ of less than about 200 μm.

In one embodiment of any of the compositions described herein, the compound of Formula I, such as a compound of Formula IA, IB, IC, ID or IE, has a particle size D₉₀ of between about 60 μm and about 100 μm.

In one embodiment of any of the compositions described herein, the compound of Formula I, such as a compound of Formula IA, IB, IC, ID or IE, has a particle size D₉₀ of between about 0.1 μm and about 200 μm.

In one embodiment of any of the compositions described herein, the compound of Formula I, such as a compound of Formula IA, IB, IC, ID or IE, has a particle size D₉₀ of between about 2.0 μm and about 150 μm.

In one embodiment of any of the compositions described herein, the compound of Formula I, such as a compound of Formula IA, IB, IC, ID or IE, represents less than about 75% w/w of the total weight of the composition, such as less than about 60% w/w or less than about 50% w/w of the total weight of the composition.

In one embodiment of any of the compositions described herein, the compound of Formula I, such as a compound of Formula IA, IB, IC, ID or IE, has a particle size D₉₀ of less than about 200 μm and the compound represents about 75% w/w or less of the total weight of the composition.

In one embodiment of any of the compositions described herein, the composition is a solid, such as a tablet or capsule. In one embodiment, at least 90% of the compound of Formula I in the composition is released within 30 minutes when tested according to USP II (paddle method) at 100 rpm and 37.0±0.5° C. in 900 mL of (i) simulated gastric fluid (without pepsin) at pH 1.2, (ii) 0.01 N HCl aqueous solution containing 1% sodium lauryl sulfate, (iii) 0.1 N HCl aqueous solution containing 1% sodium lauryl sulfate, (iv) aqueous pH 4.5 acetate solution and 1% sodium lauryl sulfate, or (v) aqueous pH 6.8 phosphate solution and 1% sodium lauryl sulfate.

In one embodiment of any of the compositions described herein, the composition is a compressed tablet comprising a compound of Formula IA or a pharmaceutically acceptable salt thereof

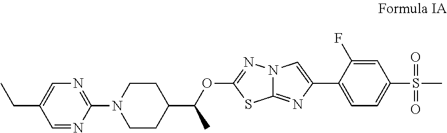 [SMILES: CCc1cnc(N2CCC([C@H](C)Oc3nn4cc(-c5ccc(S(C)(=O)=O)cc5F)nc4s3)CC2)nc1]

dispersed in
(a) a non-ionic, water dispersible surfactant (such as a mixture of lauroyl polyoxyl-32 glycerides and PEG 6000);
(b) colloidal silicon dioxide;
(c) crospovidone; and
(d) spray-dried (i) D-mannitol, (ii) xylitol, (iii) microcrystalline cellulose, (iv) crospovidone, and (v) dibasic calcium phosphate or magnesium aluminometasilicate, wherein the compound of Formula IA has a particle size D₉₀ of less than about 200 μm.

In one embodiment of any of the compositions described herein, the composition is a compressed tablet comprising a compound of Formula IA or a pharmaceutically acceptable salt thereof

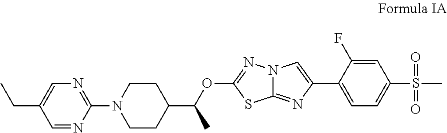 [SMILES: CCc1cnc(N2CCC([C@H](C)Oc3nn4cc(-c5ccc(S(C)(=O)=O)cc5F)nc4s3)CC2)nc1]

dispersed in
(a) a non-ionic, water dispersible surfactant (such as a mixture of lauroyl polyoxyl-32 glycerides and PEG 6000);
(b) colloidal silicon dioxide;
(c) crospovidone; and
(d) spray-dried (i) D-mannitol, (ii) xylitol, (iii) microcrystalline cellulose, (iv) crospovidone, and (v) dibasic calcium phosphate, wherein the compound of Formula IA has a particle size D₉₀ of less than about 200 μm.

In one embodiment of any of the compositions described herein, the composition is a solution or suspension. The solution or suspension may include a cyclodextrin, such as 2-hydroxypropyl beta-cyclodextrin, in addition to a compound of Formula I (such as a compound of Formula IA) or a pharmaceutically acceptable salt thereof. In one embodiment, the solution includes a sufficient amount of the cyclodextrin (such as 2-hydroxypropyl beta-cyclodextrin) to solubilize the compound of Formula I (such as a compound of Formula IA) or a pharmaceutically acceptable salt thereof. In one embodiment, the ratio of the compound of Formula IA to 2-hydroxypropyl beta-cyclodextrin in the solution or suspension is about 1:1 to about 1:20, such as about 1:5 to about 1:10.

In another aspect, the present invention relates to a process for the preparation of an oral pharmaceutical composition comprising a compound of Formula I, such as a compound of Formula IA, IB, IC, ID or IE, wherein the process comprises wet granulation, direct compression, dry granulation, solid dispersion, melt granulation, or extrusion. In one embodiment, the composition includes a therapeutically effective amount of the compound of Formula I.

In another aspect, the present invention relates to a process for the preparation of an oral pharmaceutical composition comprising a compound of Formula I, such as a compound of Formula IA, IB, IC, ID or IE, and one or more pharmaceutically acceptable excipients, the process comprising:
(a) preparing a blend or granules of a compound of Formula I, such as a compound of Formula IA, IB, IC, ID or IE, and one or more pharmaceutically acceptable excipients;
(b) compressing the product of step (a) to form tablets or filling the product of step (a) in capsules.

In another aspect, the present invention relates to a process for the preparation of an oral pharmaceutical composition comprising a compound of Formula I, such as a compound of Formula IA, IB, IC, ID or IE, and one or more pharmaceutically acceptable excipients, the process comprising:
(a) preparing a solution or a suspension comprising a compound of Formula I, such as a compound of Formula IA, IB, IC, ID or IE, a solvent, and, optionally, one or more pharmaceutically acceptable excipients;
(b) adding the solution or suspension of step (a) to one or more pharmaceutically acceptable excipients to obtain granules, or coating the solution or suspension of step (a) onto inert cores to obtain drug-coated cores;
(c) optionally, blending the granules or drug-coated cores of step (b) with one or more pharmaceutically acceptable excipients; and
(d) compressing the granules or drug-coated cores of step (b) or the blend of step (c) to form tablets or filling the granules or drug-coated cores of step (b) or the blend of step (c) in capsules.

In another aspect, the present invention relates to a method of treating diabetes or obesity comprising orally administering a composition according to any of the embodiments described herein to a patient in need thereof.

### DETAILED DESCRIPTION OF THE INVENTION

As used herein, the singular forms “a”, “an”, and “the” include plural references unless the context dictates otherwise. Thus, for example, a reference to “a method” or “a process” includes one or more methods, one or more processes, and/or steps of the type described herein and/or which will become apparent to those persons skilled in the art upon reading this disclosure.

As used herein, the terms “Compound of Formula I”, including “Compound of Formula IA-IE,” “Compound of Formula IA, IB, IC, ID and IE,” and “active ingredient” refers to a compound as described herein, as well as its pharmaceutically acceptable or therapeutically active salts, esters, amides, prodrugs, metabolites, enantiomers, polymorphs, and analogues that induce a desired pharmacological or physiological effect. Terms like “active”, “active agent”, “therapeutic agent”, “therapeutic ingredient,” “therapeutic substance,” and “active substance” may be used synonymously for “active ingredient” and/or “Compound of Formula I including “Compound of Formula IA-IE,” “Compound of Formula IA, IB, IC, ID and IE.

As used herein the term “Compound of Formula I” refers to compounds of the formula

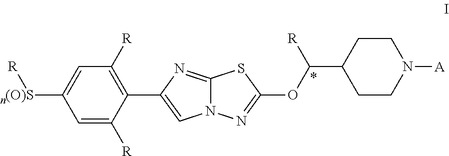

wherein
each R is independently selected from H, halogen, substituted or unsubstituted C₁-4 alkyl, and substituted or unsubstituted C₁₋₄ alkoxy;
A is selected from H, substituted or unsubstituted C₁₋₄ alkyl, substituted or unsubstituted phenyl, substituted or unsubstituted (C₁₋₄ alkyl)phenyl, substituted or unsubstituted 5-6 membered heteroaryl containing one or more heteroatoms selected from N, O, and S, substituted or unsubstituted heterocyclyl containing one or more heteroatom selected from N, O, and S, and substituted or unsubstituted C₁₋₄ alkoxycarbonyl;
wherein the substituents are selected from halogen, substituted or unsubstituted C₁₋₄ alkyl, and substituted or unsubstituted C₁₋₄ alkoxy, n=0, 1, 2, 3; and
* denotes a stereocenter.

The substituents in the aforementioned “substituted” groups cannot be further substituted. For example, when the substituent on “substituted C₁₋₄ alkyl” is “substituted C₁₋₄ alkoxy”, the substituent on “substituted C₁₋₄ alkoxy” cannot be “substituted C₁₋₄ alkyl”.

As used herein, the term “C₁₋₄ alkyl”, unless otherwise specified, refers to a straight or branched hydrocarbon chain radical consisting solely of carbon and hydrogen atoms, containing no unsaturation, having from one to four carbon atoms, and which is attached to the rest of the molecule by a single bond, e.g., methyl, ethyl, n-propyl, 1-methylethyl (isopropyl), n-butyl.

The term “C₁₋₄ alkoxy” unless otherwise specified, denotes an alkyl group as defined above attached via an oxygen linkage to the rest of the molecule. The term “substituted alkoxy” refers to an alkoxy group where the alkyl constituent is substituted (i.e., —O-(substituted alkyl) wherein the term “substituted alkyl” is the same as defined above for “alkyl”. For example “alkoxy” refers to the group-O-alkyl, including from 1 to 4 carbon atoms of a straight or branched configuration and combinations thereof attached to the parent structure through oxygen. Examples include methoxy, ethoxy, propoxy, isopropoxy, and butoxy. The term “alkoxy” also includes groups in which the alkyl group is cyclic, such as, e.g., cyclopropoxy and cyclobutoxy.

As used herein, the term “aryl”, unless otherwise specified, refers to aromatic radicals having in the range of 6 up to 20 carbon atoms such as phenyl, naphthyl, tetrahydronaphthyl, indanyl and biphenyl.

In certain embodiments of any of the formulations described herein, the Compound of Formula I is selected from compounds of Formulas IA, IB, IC, ID and IE:

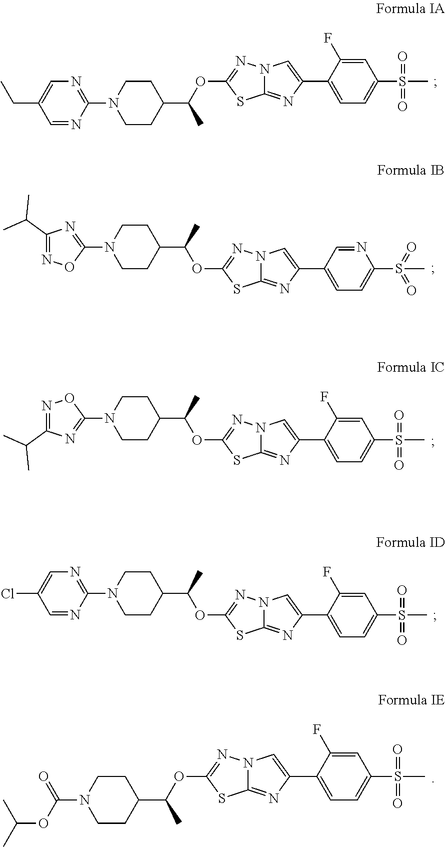

In one embodiment of any of the formulations described herein, the Compound of Formula I is a Compound of Formula IA.

As used herein, the term “about” in reference to a numerical value means that variations of 10% above or below the numerical value are within the range ascribed to the specified value.

As used herein, the term “therapeutically effective amount” means the amount or quantity of a Compound of Formula I, such as a compound selected from Compounds of Formula IA, IB, IC, ID and IE, which is sufficient to elicit a desired biological response when administered to a patient. It will be appreciated that the precise therapeutically effective amount will depend on the age and condition of the patient, the nature of the condition to be treated and will be at the ultimate discretion of the attendant physician.

As used herein, the terms “formulation,” “composition,” “oral formulation,” “oral composition,” “dosage form,” “pharmaceutical formulation,” and “pharmaceutical composition” may be used interchangeably, and include, but are not limited to, solid and liquid dosage forms, for example tablets, such as mono-layered tablets, bi-layered tablets, tri-layered tablets, multilayer tablets, caplets, minitablets, capsules, tablets in tablets, tablets in a capsule, granules in a capsule, pellets, pellets in a capsule, powders, granules, solid dispersions, drug coated inert cores, beads, spheroids, suspensions, solutions, and any other suitable dosage form suitable for oral administration. The formulations described herein include immediate release, fast release, orally disintegrating, extended release, modified release, delayed release, pulsed release formulations, and the like. The formulations may further be compounded in a drug carrier or drug delivery system, e.g., in order to improve stability and/or solubility or further improve bioavailability. The formulations may be a solid dispersion or a freeze-dried or spray-dried formulation.

As used herein, in certain embodiments, the term “stable,” as in a “stable pharmaceutical formulation,” refers to a pharmaceutical formulation comprising a Compound of Formula I according to any of the embodiments described herein in which the amount of impurity (ies) remains below FDA acceptable intake limit after exposure of the pharmaceutical formulation to 40° C.±2° C. and 75%±5% RH for a period of six months or 25° C.±2° C. and 60%±5% RH for a period of at least 12 months.

As used herein, the term “bioavailability” refers to the fraction of an administered dose of an active pharmaceutical ingredient (API) and/or active moieties, such as a Compound of Formula I as defined in any embodiment herein, which reaches the systemic circulation unchanged or in another active form. By definition, when an API and/or active moieties are administered intravenously, their bioavailability is 100%. However, when it is administered via other routes (such as orally), its bioavailability decreases (due to incomplete absorption and/or first-pass metabolism). Absolute oral bioavailability is calculated as the relative exposure of the API and/or active moieties in systemic circulation following oral administration (estimated as the area under the plasma concentration versus time curve) compared to the exposure of the API following intravenous administration.

As used herein, the term “excipient” or “pharmaceutically acceptable excipient” means a pharmacologically inactive component. Suitable pharmaceutically acceptable excipients for use in any of the formulations described herein include, but are not limited to, diluent(s)/filler(s), binder(s), disintegrant(s), glidant(s), surfactant(s), solubilizer(s), extended release or delayed release excipient(s), pH modifier(s), anti-oxidant(s), preservative(s), lubricant(s), flavoring agent(s), plasticizer(s), colorant(s), opacifier(s), carrier(s), and any combination of any of the foregoing. The excipients that are useful in preparing a dosage form are generally safe, non-toxic, and acceptable for veterinary as well as human pharmaceutical use. Reference to an excipient includes both one excipient and more than one excipient.

As used herein, the term “solid dispersion” refers to a dispersion of a Compound of Formula I (such as a compound selected from Compounds IA, IB, IC, ID and IE, in particular a Compound of Formula IA), where the Compound of Formula I exists in a solubilized, amorphous, or a mixture of amorphous and crystalline states in the formulation.

Any of the formulations described herein are stable and orally bioavailable. The Compounds of Formula I described herein have limited solubility in water.

The present invention relates to a pharmaceutical formulation comprising a therapeutically effective amount of a Compound of Formula I (such as a compound selected from Compounds IA, IB, IC, ID and IE, in particular a Compound of Formula IA) and one or more pharmaceutically acceptable excipients that are stable and orally bioavailable.

In additional embodiments of any of the formulations described herein, the Compound of Formula I (such as a compound selected from Compounds IA, IB, IC, ID and IE, in particular a Compound of Formula IA) is present in the pharmaceutical formulation in an amount of between about 50 mg and about 500 mg, such as between about 50 mg and about 500 mg.

In additional embodiments of any of the formulations described herein, the Compound of Formula I (such as a compound selected from Compounds IA, IB, IC, ID and IE, in particular a Compound of Formula IA) is present in the pharmaceutical formulation in an amount of about 100 mg, about 200 mg, about 300 mg, about 400 mg or about 500 mg.

In additional embodiments of any of the formulations described herein, the Compound of Formula I (such as a compound selected from Compounds IA, IB, IC, ID and IE, in particular a Compound of Formula IA) is amorphous, substantially crystalline (e.g., greater than 90%, or greater than 95%, greater than 99% crystalline) or is crystalline.

In additional embodiments of any of the formulations described herein, the Compound of Formula I (such as a compound selected from Compounds IA, IB, IC, ID and IE, in particular a Compound of Formula IA) is amorphous.

In additional embodiments of any of the formulations described herein, the Compound of Formula I (such as a compound selected from Compounds IA, IB, IC, ID and IE, in particular a Compound of Formula IA) is substantially crystalline (e.g., greater than 90%, greater than 95%, or greater than 99% crystalline) or is crystalline.

Suitable diluents/fillers for use in any of the formulations described herein include, but are not limited to, organic and inorganic diluents, such as, for example, celluloses and cellulose derivatives, starches and modified starches, polyols, carbohydrates, inorganic phosphates, carbonates, silicates, sulfates, and any combination of any of the foregoing.

In certain embodiments, the diluents/fillers are selected from microcrystalline cellulose, silicified microcrystalline cellulose, polyols and sugar alcohols selected from, but not limited to, alditols, mannitol, D-mannitol, meglumine, xylitol, isomalt, sorbitol, lactitol, pentitol, arabitol, ribitol, galactitol, erythritol, glycerol, and the like; carbohydrates selected from, but not limited to, monosaccharides, oligosaccharides polysaccharides, dextrins, maltodextrin, pullulan, arabinose, dextrose, dextrates, lactose, sucrose, sucralose, saccharin, fructose, maltose, trehalose, psicose, tagatose, sorbose, cellulose derivatives, cellobiose, starches, modified starches, sucrose fatty acid esters, and the like; inorganic diluents selected from, but not limited t,o dibasic calcium phosphate, tribasic calcium phosphate, calcium carbonate, kaolin, calcium sulfate, and the like; any any combination of any of the foregoing.

Diluents may include co-processed excipients such as D-mannitol and starch (Pearlitol® Flash), lactose and microcrystalline cellulose (Cellactose®), D-mannitol, xylitol, microcrystalline cellulose, Crospovidone, dibasic calcium phosphate anhydrous (F-MELT® type C). F-MELT® is available in two grades, F-MELT® Type C and F-MIELT® Type M. Type C contains dibasic calcium phosphate anhydrous (Fujicalin®) and Type M contains magnesium aluminometasilicate (Neusilin®). When present, a filler may be employed in an amount ranging from about 1% to about 80% by weight of the pharmaceutical formulation, such as between about 1% to about 60% by weight of the pharmaceutical formulation, such as between about 1.

Suitable binders for use in any of the formulations described herein include, but are not limited to, natural and synthetic polymers, natural and synthetic gums, hydrocolloids, celluloses and cellulose derivatives, starches and modified starches, carbohydrates, vinyl polymers, and any combination of any of the foregoing. For example, in certain embodiments, the binder is selected from polyvinylpyrrolidone (PVP), e.g., PVP K30 or PVP K90, polyvinyl acetate, polyvinyl alcohol, polyethylene glycols (PEG), e.g., PEG 400, PEG 4000, PEG 6000, copolymers of PEG, polyvinyl alcohol, polyvinylacetate, and PVP, hydroxypropylmethyl cellulose, hydroxypropyl cellulose (in certain embodiments both the hydroxypropylmethyl cellulose and the hydroxypropyl cellulose are of medium to high viscosity, such as viscosity grades 3 or 6 cps), copovidone, maltodextrins, pregelatinized starch, microcrystalline cellulose, acacia, alginic acid, tragacanth, gelatin, liquid glucose, corn starch, sugars, ethyl cellulose, methylcellulose, hydroxyethyl cellulose, carboxymethyl cellulose, sodium alginate, and any combination of any of the foregoing. When present, a binder may be employed in an amount ranging from about 0.1% to about 25% by weight of the pharmaceutical formulation.

Suitable disintegrants for use in any of the formulations described herein include, but are not limited to, natural and synthetic polymers, natural and synthetic gums, celluloses and cellulose derivatives, starches and modified starches etc. For example, in certain embodiments, the disintegrant is selected from low-substituted hydroxypropyl cellulose, cross-linked polyvinylpyrrolidone, polyvinylpyrrolidone, corn starch, cross-linked sodium carboxymethylcellulose, sodium carboxymethylcellulose, microcrystalline cellulose, sodium starch glycolate, crosslinked CMC (Ac-Di-Sol), sodium carboxymethyl starch, ion-exchange resins, formalin-casein, used either alone or combinations thereof. When present, a disintegrant may be employed in an amount ranging from about 0.1% to about 20% by weight of the pharmaceutical formulation.

Suitable lubricants for use in any of the formulations described herein include, but are not limited to, zinc stearate, magnesium stearate, sodium stearyl fumarate, calcium stearate, stearic acid, colloidal silica, aluminium or calcium silicate, stearic acid, cutina, PEG 4000-8000, talc and any combination of any of the foregoing. When present, a lubricant may be employed in an amount ranging from about 0.01% to about 10%, preferably from about 0.1% to about 5% by weight of the pharmaceutical formulation.

Suitable glidants for use in any of the formulations described herein include, but are not limited to, colloidal silicon dioxide (e.g., Aerosil 200), mesoporous silica, stearates, magnesium trisilicate, powdered cellulose, starch, talc and any combination of any of the foregoing. When present, a glidant may be employed in an amount ranging from about 0.01% to about 10%, such as from about 0.1% to about 5% by weight of the pharmaceutical formulation.

Suitable solubilizing agents for use in any of the formulations described herein include, but are not limited to, surfactants, cyclic oligosaccharides/cyclodextrins, phospholipids, and any combination of any of the foregoing. The solubilizing agent(s) may be employed in an amount ranging from about 0.01% to about 25% by weight of the pharmaceutical formulation, such as from about 1% to about 20% by weight of the pharmaceutical formulation.

Suitable surfactants for use in any of the formulations described herein include anionic, cationic, non-ionic and amphoteric surfactants known to the person skilled in the art. Suitable surfactants include, but are not limited to, sodium docusate, sodium lauryl sulfate, sodium stearyl fumarate, Tweens® and Spans (PEO modified sorbitan monoesters and fatty acid sorbitan esters), poloxamers, polysorbates, alkylaryl polyethers, polyoxyethyleneglycol alkyl ethers, polyoxyls, and any combination of any of the foregoing. Poloxamers are nonionic triblock copolymers composed of a central hydrophobic chain of polyoxypropylene (poly (propylene oxide)) flanked by two hydrophilic chains of polyoxyethylene (poly(ethylene oxide)). Polysorbates are oily liquids derived from ethoxylated sorbitan esterified with fatty acids. Polyoxyls are a mixture of mono- and diesters of stearate and polyoxyethylene diols. In certain embodiments suitable surfactants include, but are not limited to, poloxamer 188 (such as Pluronic® F-68) and poloxamer 407 (such as Pluronic® F127); polysorbate 20, polysorbate 60, polysorbate 80, tyloxapol, Brij® 35, Brij® 78, Brij® 98 and Brij® 700, Span® 20, Span® 40, Span® 60, Span® 80; and polyoxylspolyoxyl 40 stearate, polyoxyl 30 castor oil, polyoxyl 35 castor oil, and polyoxyl 40 hydrogenated castor oil; Hydrogenated Castor oil (or PEG (40 Hydrogenated castor oil) (HCO-40), Kolliphore EL (Cremophor EL), Cremophor RH 40, Cremophor RH 60, d-α-tocopherol polyethylene glycol 1000 succinate, Solutol HS 15, sorbitan monooleate, Labrafil M-1944CS, Labrafil M-2125CS, Labrasol, Gellucire 44/14, Softigen 767, and mono- and di-fatty acid esters of PEG 300, 400, or 1750, and any combination of any of the foregoing. When present, a non-ionic surfactant may be employed in an amount ranging from about 0.01% to about 25% by weight of the pharmaceutical formulation.

Suitable solubilizers for use in any of the formulations described herein include, but are not limited to, cyclic oligosaccharides/cyclodextrins including, without limitation, α-cyclodextrin, β-cyclodextrin, hydroxypropyl-β-cyclodextrin, and sulfobutylether-β-cyclodextrin, cyclodextrins-2-HP25, ionically charged (e.g. anionic) β-cyclodextrins with or without a butyrated salt (Captisol®), hydroxypropyl-gamma-cyclodextrin, gamma cyclodextrin, phospholipids including, without limitation, hydrogenated soy phosphatidyl choline, distearoyl phosphatidyl glycerol, 1-α-dimyristoylphosphatidylcholine, 1-α-dimyristoyl phosphatidyl glycerol, polyethylene glycol (PEG), and any combination of any of the foregoing. When present, a cyclodextrin may be employed in an amount ranging from about 0.01% to about 25%, by weight of the pharmaceutical formulation.

Suitable pH modifiers for use in any of the formulations described herein include, but are not limited to, an acid selected from an organic or inorganic acid such as ascorbic acid, fumaric acid, citric acid, malic acid, succinic acid, adipic acid, maleic acid, lactic acid, hydrochloric acid, nitric acid, phosphoric acid, sulfuric acid, and the like or a base selected from organic bases such as pyridine, alkanamines, such as methylamine, diisopropylamine, diisopropylethylamine triethylamine, dimethylamine, trimethyl amine, pyridine, imidazole, histidine, guanidine, poly ethyleneimine, poly (vinylpyridine), diethanolamine, triethanolamine, tris(hydroxymethyl)aminomethane (Tris), sodium glycine, 1-methylimidazole, 2-methylimidazole, and 4(5)-methylimidazole, and 1,2-diaminoethane, 2-(bis(2-hydroxyethyl)amino)-2-(hydroxymethyl) propane-1,3-diol, sodium lysine, sodium histidine, and sodium arginine, polyvinyl imidazole, and copolymers thereof (e.g., a copolymer of poly ethyleneimine and one or more of poly (vinylpyridine) and polyvinylimidazole, or a copolymer of poly (vinylpyridine) with polyvinyl imidazole) or inorganic bases such as sodium carbonate, sodium bicarbonate, potassium bicarbonate, lithium carbonate, potassium carbonate, calcium carbonate, magnesium carbonate, magnesium oxide, sodium citrate, sodium hydroxide, potassium hydroxide, ammonium salts, and any combination of any of the foregoing. In one embodiment, when present, a pH modifier may be employed in an amount ranging from about 0.1% to about 10%, by weight of the pharmaceutical formulation.

Suitable antioxidants for use in any of the formulations described herein include, but are not limited to, natural and synthetic compounds, organic and inorganic acids etc. In certain embodiments, the antioxidant is selected from ascorbic acid, tartaric acid, succinic acid, maleic acid, fumaric acid, citric acid, malic acid, lactic acid, benzenesulfonic acid, oxalic acid, triphenylacetic acid, 1-hydroxy-2-naphthoic acid, 3-hydroxy-2-naphthoic acid, sodium ascorbate, alpha-tocopherol, butylated hydroxytoluene, butylated hydroxyanisole, gentisic acid (2,5-dihydroxy benzoic acid), acetyl cysteine, ascorbyl palmitate, cysteine, dithiothreitol, thioglycerol, thiourea, caffeic acid, propyl gallate, ferulic acid, sodium pyrosulfite, edetic acid, edetate salts, 2, 6-di-tert-butyl p-cresol, gallic acid and esters thereof, nordihydroguaiaretic acid, benzoic acid, benzoates, sorbic acid, sorbates, guaiacol ester, tea polyphenol, curcumin, chlorogenic acid, methionine, proline, biflavonoids, superoxide dismutase, silymarin, grape skin/seed extract, melanin, rosemary extract, sodium sulfite, sodium thiosulfate, sodium bisulfite, sodium metabisulfite, tert-butylhydroquinone, sodium citrate, magnesium ascorbyl phosphate, sodium ascorbyl phosphate, 3,4-dihydroxybenzoic acid, butylated hydroxybenzoic acid and salts thereof, erythorbic acid and sodium salts thereof, sorbic acid and salts thereof, sodium formaldehyde sulfoxylate, glutathione, lipoic acid, dihydroxy fumaric acid, and the like. In one embodiment, when present, an antioxidant may be employed in an amount ranging from about 0.1% to about 10%, by weight of the pharmaceutical formulation.

Suitable extended release or delayed release excipients for use in any of the formulations described herein include, but are not limited to, hydrophilic or hydrophobic agents, natural and synthetic polymers, natural and synthetic gums, In certain embodiments, the extended release or delayed release excipient is selected from polyvinyl acetate, cellulose acetate, cellulose acetate butyrate, cellulose acetate propionate, ethyl cellulose, a fatty acid, a fatty acid ester, an alkyl alcohol, a wax, xanthan gum, gellan gum, shellac, rosin, zein (prolamine from corn), povidone, kollidon SR (polyvinyl acetate and povidone), a poly(meth)acrylate, poly(ethylene oxide), polyuronic acid salts, cellulose ethers, xanthan gum, tragacanth gum, gum karaya, guar gum, acacia, gellan gum locust bean gum, alkali metal salts of alginic acid or pectic acid, sodium alginate, potassium alginate, ammonium alginate, polyethylene oxide, carbomer homopolymer, hydroxypropyl cellulose, hydroxy ethyl cellulose, hydroxypropyl methyl cellulose, carboxyvinyl polymers, polymerized gelatin, shellac, methacrylic acid copolymer type C NF, cellulose butyrate phthalate, cellulose hydrogen phthalate, cellulose propionate phthalate, polyvinyl acetate phthalate (PVAP), cellulose acetate phthalate (CAP), cellulose acetate trimellitate (CAT), hydroxypropyl methylcellulose phthalate, hydrogenated castor oil, stearic acid, hydrogenated vegetable oil, glyceryl behenate, glyceryl monostearate, hydroxypropyl methylcellulose acetate, dioxypropyl methylcellulose succinate, carboxymethyl ethyl cellulose (CMEC), hydroxypropyl methylcellulose acetate succinate (HPMCAS), and acrylic acid polymers and copolymers like methyl acrylate, ethyl acrylate, methyl methacrylate and/or ethyl methacrylate with copolymers of acrylic and methacrylic acid esters (Eudragit NE, Eudragit RL, Eudragit RS) and any combination of any of the foregoing. When present, a polymer may be employed in an amount ranging from about 0.1 to about 50% by weight of the formulation, such as from about 10 to about 50% or about 10 to about 40% by weight of the formulation.

Suitable inert cores for use in any of the formulations described herein include, but are not limited to, sugar, sucrose, sorbitol, xylitol, mannitol, lactose, dicalcium phosphate, microcrystalline cellulose, maltodextrin, starch, a hydrophilic cellulosic polymer, or a crosslinked hydrophilic synthetic polymer or can be selected from any filler or diluent, or any combination of any of the foregoing.

Solvents that may be used to prepare a solution or a dispersion (e.g., suspension) of a Compound of Formula I (such as a compound selected from Compounds IA, IB, IC, ID and IE, in particular a Compound of Formula IA) may be an aqueous solvent, a non-aqueous solvent (e.g., an organic solvent, an inorganic solvent), or any combination thereof.

Suitable aqueous solvents include, for example, purified water, water for injection, bacteriostatic water for injection, sterile water, isotonic saline water, buffered aqueous solution. Suitable non-aqueous solvents include organic solvents or inorganic solvents. Suitable organic solvents include, but are not limited to, acetic acid, methylene chloride, to monohydric or polyhydric alcohols, isopropyl alcohol, ethanol, glycerin, propylene glycol, acetone, acetonitrile, benzene, 1-butanol, 2-butanol, 2-butanone, t-butyl alcohol, carbon tetrachloride, chlorobenzene, chloroform, cyclohexane, 1,2-dichloroethane, dehydrated alcohol, diethylene glycol, diethyl ether, diglyme (diethylene glycol dimethyl ether), 1,2-dimethoxy-ethane (glyme, DME), dimethyl formamide (DMF), dimethyl sulfoxide (DMSO), 1,4-dioxane, ethyl acetate, ethylene glycol, heptane, Hexamethylphosphoramide (HMPA), Hexamethylphosphorous triamide (HMPT), hexane, methanol, methyl t-butyl ether (MTBE), N-methyl-2-pyrrolidinone (NMP), nitromethane, pentane, Petroleum ether (ligroine), 1-propanol, 2-propanol, pyridine, tetrahydrofuran (THF), toluene, triethyl amine, o-xylene, m-xylene, benzyl alcohol, glycofurol, solketal, glycerol formal, and any combination thereof. Suitable inorganic solvent include, but not limited to, water, aqueous solutions containing special additives (surfactants, detergents, PH buffers, inhibitors), liquid anhydrous Ammonia, concentrated sulfuric acid, sulfuryl chloride fluoride, liquid sulfur dioxide, sulfuryl chloride and, phosphoryl chloride, dinitrogen tetroxide, antimony trichloride, bromine pentafluoride, hydrogen fluoride, and any combination thereof.

Suitable coating agents for use in any of the formulations described herein include, but are not limited to, immediate release, extended release, and delayed release coatings. Suitable examples include, but are not limited to, Shellac, cellulose acetate phthalate (CAP), polyvinylacetate phthalate (PVAP), hyroxylpropyl cellulose, hyroxypropyl methylcellulose (HPMC), acrylates, phthalates, and Zein (a corn protein derivative), hydroxypropyl methyl cellulose, methyl hydroxyethyl cellulose, ethylcellulose, povidone, polyvinyl acetate, polyvinyl alcohol, Opadry, and any combination of any of the foregoing.

In one embodiment of any of the formulations described herein, the Compound of Formula I (such as a compound selected from Compounds IA, IB, IC, ID and IE, in particular a Compound of Formula IA) has a D₉₀ particle size of less than about 200 μm, such as less than about 150 μm, less than about 100 μm, less than about 75 μm, less than about 50 μm, less than about 25 μm, or less than about 10 μm.

In one embodiment of any of the formulations described herein, the Compound of Formula I (such as a compound selected from Compounds IA, IB, IC, ID and IE, in particular a Compound of Formula IA) has a D₅₀ particle size of about 5 to about 20 μm. In one embodiment of any of the formulations described herein, the Compound of Formula I (such as a compound selected from Compounds IA, IB, IC, ID and IE, in particular a Compound of Formula IA) has a D₁₀ particle size of about 1 to about 10 μm.

In one embodiment, the present invention relates to an oral pharmaceutical formulation comprising a therapeutically effective amount of Compound of Formula I (such as a compound selected from Compounds IA, IB, IC, ID and IE, in particular a Compound of Formula IA) wherein the compound of Formula I represents about 75% or less of the weight of the composition, such as about 50% or less of the weight of the composition, or about 25% or less of the weight of the composition

In one embodiment of any of the oral formulations described herein, the oral formulation is a tablet or a capsule. In one embodiment, the oral formulation is an immediate-release tablet. In certain embodiments of any of the oral formulations described herein, the weight of the dosage form (e.g., tablet, such as an immediate-release tablet) is about 100 mg to about 2000 mg.

In one embodiment, the present invention relates to a process for the preparation of an oral pharmaceutical formulation comprising a Compound of Formula I (such as a compound selected from Compounds IA, IB, IC, ID and IE, in particular a Compound of Formula IA) wherein the process comprises dry granulation, wet granulation, direct compression, melt granulation, extrusion, or a solid dispersion process.

In one embodiment, the present invention relates to a process for preparation of an oral pharmaceutical formulation of a Compound of Formula I (such as a compound selected from Compounds IA, IB, IC, ID and IE, in particular a Compound of Formula IA), and one or more pharmaceutically acceptable excipients, the process comprising:
(1) preparing a blend (a mixture) or granules of a Compound of Formula I (such as a compound selected from Compounds IA, IB, IC, ID and IE, in particular a Compound of Formula IA) and one or more pharmaceutically acceptable excipients; and
(2) compressing the blend or granules to form tablets or filling the blend or granules in capsules.

In one embodiment, the present invention relates to a process (e.g., a direct compression process) for the preparation of an oral pharmaceutical formulation comprising a Compound of Formula I (such as a compound selected from Compounds IA, IB, IC, ID and IE, in particular a Compound of Formula IA) and one or more pharmaceutically acceptable excipients, the process comprising:
(1) premixing the Compound of Formula I (such as a compound selected from Compounds IA, IB, IC, ID and IE, in particular a Compound of Formula IA) and a portion (such as a portion >50%) of the one or more pharmaceutically acceptable excipients (e.g., in a mixer) to obtain a pre-mixture;
(2) optionally dry screening the pre-mixture through a screen (e.g., in order to segregate cohesive particles and to improve content uniformity);
(3) mixing the pre-mixture of step (1) or (2) (e.g., in a mixer), optionally adding the remained of the one or more pharmaceutically acceptable excipients to the mixture;
(4) tableting the final mixture of step (3) (e.g., by compressing it on a suitable tablet press) to produce tablet cores;
(5) optionally film-coating the tablet cores of step (4) with a film coat.

In one embodiment, the present invention provides process (e.g., a dry granulation process) for the preparation of an oral pharmaceutical formulation comprising a Compound of Formula I (such as a compound selected from Compounds IA, IB, IC, ID and IE, in particular a Compound of Formula IA) and one or more pharmaceutically acceptable excipients, the process comprising:
(1) mixing the Compound of Formula I (such as a compound selected from Compounds IA, IB, IC, ID and IE, in particular a Compound of Formula IA) with a portion (such as less than 100% or 100%) of the one or more pharmaceutically acceptable excipients (e.g., in a mixer);
(2) compacting the mixture of step (1) (e.g., on a suitable roller compactor) (e.g., to obtain ribbons);
(3) reducing the compacted mixture (e.g., ribbons) obtained during step (2) to granules (e.g., by suitable milling and/or sieving steps);
(4) optionally mixing the granules of step (3) with the remaining one or more pharmaceutically acceptable excipients (e.g. in a mixer) to obtain a final mixture;
(5) tabletting the granules of step (3) or the final mixture of step (4) (e.g., by compressing it on a suitable tablet press) to produce tablet cores;
(6) optionally film-coating the tablet cores of step (5) with a film coat.

In one embodiment, the present invention relates to a process (e.g., a wet granulation process for the preparation of an oral pharmaceutical formulation comprising a Compound of Formula I (such as a compound selected from Compounds IA, IB, IC, ID and IE, in particular a Compound of Formula IA) and one or more pharmaceutically acceptable excipients, the process comprising:
(1) premixing the Compound of Formula I (such as a compound selected from Compounds IA, IB, IC, ID and IE, in particular a Compound of Formula IA) and a portion (such as a portion >50%) of the one or more pharmaceutically acceptable excipients (e.g., in a mixer) to obtain a pre-mixture;
(2) granulating the pre-mixture of step (1) (e.g., by adding a granulation liquid);
(3) drying the granules of step (2) (e.g., in a fluidized bed dryer or a drying oven);
(4) optionally dry sieving the dried granules of step (3);
(5) mixing the dried granules of step (2) or step (4) with the remaining one or more pharmaceutically acceptable excipients (e.g., in a mixer) to obtain a final mixture;
(6) tableting the final mixture of step (5) (e.g., by compressing it on a suitable tablet press) to produce tablets cores or filling the final mixture of step (5) in capsules;
(7) optionally film-coating the tablet cores of step (6) with a film coat.

In one embodiment, the present invention relates to a process for preparation of an oral pharmaceutical composition comprising a Compound of Formula I (such as a compound selected from Compounds IA, IB, IC, ID and IE, in particular a Compound of Formula IA) and one or more pharmaceutically acceptable excipients, the process comprising:
(1) preparing a solution or suspension of the Compound of Formula I (such as a compound selected from Compounds IA, IB, IC, ID and IE, in particular a Compound of Formula IA), optionally with one or more pharmaceutically acceptable excipients in a suitable solvent;
(2) spraying the solution or suspension of step (1) onto one or more pharmaceutically acceptable excipients to obtain granules or coating the solution or suspension of step (1) onto inert cores to obtain drug-coated cores;
(3) optionally blending the granules or drug-coated cores of step (2) with one or more pharmaceutically acceptable excipients;
(4) compressing the granules or drug-coated cores of step (2) or the blend of step (3) to form tablets or filling the granules or drug-coated cores of step (2) or the blend of step (3) in capsules.

In some embodiments, the formulations described herein may be granulated prior to being compacted. The formulations may comprise an intragranular part and an extragranular part, wherein the intragranular part has been granulated and the extragranular part has been added after granulation. In some embodiments, the intragranular part may comprise the Compound of Formula I (such as a compound selected from Compounds IA, IB, IC, ID and IE, in particular a Compound of Formula IA) and one or more pharmaceutically acceptable excipients. In some embodiments, the extragranular part may comprise the Compound of Formula I (such as a compound selected from Compounds IA, IB, IC, ID and IE, in particular a Compound of Formula IA) and one or more pharmaceutically acceptable excipients. The extragranular part may comprise a filler, a binder, a disintegrant, a lubricant, a glidant, or any combination thereof.

In one embodiment, the present invention provides a process for preparation of an oral pharmaceutical composition comprising a Compound of Formula I (such as a compound selected from Compounds IA, IB, IC, ID and IE, in particular a Compound of Formula IA), the process comprising an extrusion process. Extrusion may be wet extrusion or melt extrusion. Wet extrusion involves preparing a wet mass of a Compound of Formula I (such as a compound selected from Compounds IA, IB, IC, ID and IE, in particular a Compound of Formula IA) and one or more pharmaceutically acceptable excipients followed by extrusion of the wet mass to form extruded pellets. Melt extrusion involves preparing a molten solidified mass of a Compound of Formula I (such as a compound selected from Compounds IA, IB, IC, ID and IE, in particular a Compound of Formula IA) and one or more pharmaceutically acceptable excipients followed by extrusion of the molten solidified mass to form extruded pellets.

Another embodiment of the present invention relates to an amorphous solid dispersion comprising a Compound of Formula I (such as a compound selected from Compounds IA, IB, IC, ID and IE, in particular a Compound of Formula IA), and one or more pharmaceutically acceptable excipients.

In certain embodiments, an immediate release dosage form as disclosed herein releases more than about 75% of the active ingredient, e.g., a Compound of Formula I (such as a compound selected from Compounds IA, IB, IC, ID and IE, in particular a Compound of Formula IA) contained therein within a period of time selected from less than one hour, less than 45 minutes, less than 30 minutes, less than 15 minutes, and less than 10 minutes after administration.

In additional embodiments of any of the processes described herein, the process further comprises purging a solution or suspension formulation with an inert gas to reduce the oxygen concentration.

In another aspect, the present invention relates to a kit (such as a package or storage container) comprising a formulation according to any of the embodiments described herein. In certain embodiments, the package or container comprises a material that blocks oxygen or is impervious to oxygen. In certain embodiments, the package or container comprises an oxygen scavenger. In certain embodiments, the package or container comprises a desiccant or an oxygen absorber.

In another embodiment of any of the formulation described herein comprising a Compound of Formula I (such as a compound selected from Compounds IA, IB, IC, ID and IE, in particular a Compound of Formula IA), the formulation is stable when stored for at least one month, at least three months, at least six months at 40° C.±2° C. and 75%±5% relative humidity (RH).

In another embodiment of any of the formulation described herein comprising a Compound of Formula I (such as a compound selected from Compounds IA, IB, IC, ID and IE, in particular a Compound of Formula IA), the formulation is stable when stored for at least one month, at least three months, at least six months or at least 12 months at 25° C.±2° C. and 60%±5%.

In another embodiment of any of the formulation described herein comprising a Compound of Formula I (such as a compound selected from Compounds IA, IB, IC, ID and IE, in particular a Compound of Formula IA), the formulation contains impurities below FDA acceptable daily intake limit. For any of the formulation described herein contains less about 5% or less, about 4% or less, about 3% or less, about 2% or less, or about 1% or less when stored at 40° C.±2° C. and 75%±5% RH for a period of at least six months or at 25° C.±2° C. and 60%±5% RH for a period of at least twelve months.

In another aspect, the present invention relates to an oral formulation according to any of the embodiments described herein for use in medicine, such as in the treatment of cardiovascular and metabolic disorders, including, but not limited to, diabetes mellitus, non-alcoholic fatty liver disease (NAFLD), non-alcoholic Steatohepatitis (NASH), dyslipidaemias, and related disorders thereto.

In another aspect, the present invention relates to a method of treating cardiovascular and metabolic disorders, including, but not limited to, diabetes mellitus, non-alcoholic fatty liver disease (NAFLD), non-alcoholic Steatohepatitis (NASH), dyslipidaemias, and related disorders thereto, the method comprising administering to a patient in need thereof an oral formulation according to any of the embodiments described herein

The present invention also relates to an oral formulation according to any of the embodiments described herein for use as a medicament. In certain embodiments of any of the uses and methods described herein, the formulation may be used for the following medical treatments (such as those related to diabetes):
(i) prevention and/or treatment of all forms of diabetes, such as hyperglycemia, type 2 diabetes, impaired glucose tolerance, type 1 diabetes, non-insulin-dependent diabetes, MODY (maturity-onset diabetes of the young), gestational diabetes, and/or for reduction of HbA1C;
(ii) delaying or preventing diabetic disease progression, such as progression in type 2 diabetes, delaying the progression of impaired glucose tolerance (IGT) to insulin-requiring type 2 diabetes, and/or delaying the progression of non-insulin requiring type 2 diabetes to insulin-requiring type 2 diabetes;
(iii) improving β-cell function, such as decreasing β-cell apoptosis, increasing β-cell function and/or B-cell mass, and/or restoring glucose sensitivity to β-cells;
(iv) prevention and/or treatment of cognitive disorders;
(v) prevention and/or treatment of eating disorders, such as obesity, e.g. by decreasing food intake, reducing body weight, suppressing appetite, inducing satiety; treating or preventing binge eating disorder, bulimia nervosa, and/or obesity induced by administration of an antipsychotic or a steroid; reduction of gastric motility; and/or delayed gastric emptying;
(vi) prevention and/or treatment of diabetic complications, such as neuropathy, including peripheral neuropathy; nephropathy; or retinopathy;
(vii) improving lipid parameters, such as prevention and/or treatment of dyslipidemia, lowering total serum lipids; lowering HDL; lowering small, dense LDL; lowering VLDL: lowering triglycerides; lowering cholesterol; increasing HDL; lowering plasma levels of lipoprotein a (Lp(a)) in a human; inhibiting generation of apolipoprotein an (apo(a)) in vitro and/or in vivo;
(viii) prevention and/or treatment of cardiovascular diseases, such as syndrome X; atherosclerosis; myocardial infarction; coronary heart disease; stroke, cerebral ischemia; an early cardiac or early cardiovascular disease, such as left ventricular hypertrophy; coronary artery disease; essential hypertension; acute hypertensive emergency; cardiomyopathy; heart insufficiency; exercise tolerance; chronic heart failure; arrhythmia; cardiac dysrhythmia; syncopy; atheroschlerosis; mild chronic heart failure; angina pectoris; cardiac bypass reocclusion; intermittent claudication (atherosclerosis obliterans); diastolic dysfunction; and/or systolic dysfunction;
(ix) prevention and/or treatment of gastrointestinal diseases, such as inflammatory bowel syndrome; small bowel syndrome, or Crohn's disease; dyspepsia; and/or gastric ulcers;
(x) prevention and/or treatment of critical illness, such as treatment of a critically ill patient, a critical illness poly-nephropathy (CIPNP) patient, and/or a potential CIPNP patient; prevention of critical illness or development of CIPNP; prevention, treatment and/or cure of systemic inflammatory response syndrome (SIRS) in a patient; and/or for the prevention or reduction of the likelihood of a patient suffering from bacteraemia, septicaemia, and/or septic shock during hospitalisation; and/or
(xi) prevention and/or treatment of Non-alcoholic fatty liver disease (NAFLD) such as Non-alcoholic fatty liver (NAFL), Non-alcoholic Steatohepatitis (NASH); and/or
(xii) prevention and/or treatment of polycystic ovary syndrome (PCOS).

In one aspect, the present invention relates to a method of treating diabetes or obesity comprising administering to a patient in need thereof an oral formulation according to any of the embodiments described herein. In certain embodiments, the formulation is a orally administered tablet, such as an orally administered immediate release tablet.

According to another embodiment, any of the formulations described herein may further comprise one or more additional therapeutic agents. Alternatively, any of the formulations described herein may be administered to a patient in need thereof in combination (sequentially or concomitantly) with one or more other additional agents.

For example, the one or more other additional agents may be an antidiabetic drug, such as, but not limited to, SGLT2 inhibitors, DPP IV inhibitors, biguanides, thiazolidinediones, sulfonylureas, GLP-1 agonists, angiotensin receptor blockers, anti-anginal agents, anti-arrhythmic agents, anti-hypertensive, beta adrenergic, calcium channel blockers, diuretics and other cardiovascular drugs, and any combination of any of the foregoing.

The one or more other additional agents may be a biguanide, such as metformin; a thiazolidinedione selected from the group comprising of, but not limited to, pioglitazone, rosiglitazone, troglitazone, englitazone, or darglitazone; a sulfonylurea selected from the group comprising of, but not limited to, glimepride, gliclazide, glipizide, tolbutamide, glubornuride, gliquidone, Glyburide; a glinide selected from the group comprising of, but not limited to nateglinide, repaglinide; an α-Glucosidase inhibitor selected from the group comprising of, but not limited to acarbose, miglitol, and voglibose; a SGLT-2 inhibitor selected from the group comprising of, but not limited to, dapagliflozin, empagliflozin, sotagliflozin, canagliflozin, luseogliflozin, tofogliflozin, ipragliflozin, ertugliflozin, atigliflozin, or remogliflozin; a DPP-4 selected from the group comprising of, but not limited to, sitagliptin, vildagliptin, saxagliptin, carmegliptin, gosogliptin, alogliptin, linagliptin, melogliptin, gemigliptin, anagliptin, teneligliptin, trelagliptin, dutogliptin, evogliptin, omarigliptin or a pharmaceutically acceptable salt of any of the foregoing.

While selected embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. Unless defined otherwise, all technical and scientific terms used herein have the same meaning as is commonly understood by one of skill in the art to which this invention belongs. All patents and publications referred to herein are incorporated by reference.

### EXAMPLES

The invention now will be described in particularity with the following illustrative examples. However, the scope of the present invention is not intended to be, and shall not be, limited to the exemplified embodiments below:

Particle size analysis was performed using a Malvern particle size analyzer (Mastersizer 3000, Malvern Instrument Ltd).

### Sample Preparation

Weigh accurately 100 mg of sample in 100 ml beaker. Add 3 drops of Tween-80 and 1 mL of water and make a paste with the help of glass rod. Then add 10 ml water and sonicate externally for 10 seconds with continued shaking.

### Instrument Parameters

| Dispersant | Water |
|---|---|
| Dispersant Refractive Index | 1.330 |
| Level Sensor Threshold | 100 |
| Particle Type | Non spherical |
| Scattering Model | Mie |
| Material Name | Compound of Formula I |
| | (e.g., compound of Formula IA) |
| Particle Refractive Index | 0.250 |
| Particle Absorption Index | 0.001 |
| Particle Density | 0.85 g/cm³ |
| Background measurement duration | 6 seconds |
| Sample measurement duration | 6 seconds |
| Delay between measurements | 0 seconds |
| Pre-measurement delay | 0 seconds |
| Obscuration Limit | 10-20% |
| Stirrer RPM | 2000 |
| Analysis Model | General purpose |
| Analysis sensitivity | Normal |
| Fine Mode | Off |

### Procedure

After cleaning, initialize the system and measure background. Add sample to reach obscuration between 10%-20% and wait for stable obscuration. Start the analysis or run the standard operating procedure as per given instrumental parameters. Annalize the sample in duplicate and report the average result (instrument average) of two preparations at D₁₀, D₅₀, and D₉₀.

Chemical purity was determined by high pressure liquid chromatography (HPLC).

### Example 1

| Ingredients | Quantity (% w/w of Total Formulation) |
|---|---|
| Compound of Formula I | 1-75% |
| (e.g., Formula IA) | |
| Diluent | 10-80% |
| Binder | 0-20% |
| Disintegrant | 0-20% |
| Glidant | 0.01-10% |
| Lubricant | 0.01-10% |
| Optional Film Coating | 1-20% weight gain |

### Example 2

| Ingredient | Quantity (% w/w of Total Formulation) |
|---|---|
| Compound of Formula I | 1-50 |
| (e.g., Formula IA) | |
| Microcrystalline Cellulose | 1-95 |
| Colloidal Silicon Dioxide | 0.1-20 |
| Magnesium Stearate | 0.01-10 |
| Film Coating | 0.1-10 |

### Process

1. Sift the compound of Formula 1 and microcrystalline cellulose through #40 mesh.

2. Sift the blend of Step 1 with colloidal silicon dioxide through a #40 mesh.

3. Mix the blend of Step 2 in a blender for 10-20 minutes.

4. Lubricate the blend of Step 3 in a blender for 3-7 minutes with magnesium stearate.

5. Compress the blend of step 4 on a compression machine to obtain compressed tablets.

6. Coat the tablets in a coating machine with Opadry white (weight build-up 2-4%).

### Example 3

| Ingredient | Quantity (% w/w of Total Formulation) |
|---|---|
| Compound of Formula 1 | 21.81 |
| (e.g., Formula IA) | |
| Microcrystalline Cellulose | 67.36 |
| Croscarmellose Sodium | 6.42 |
| Colloidal Silicon Dioxide | 0.98 |
| Sodium Stearyl Fumarate | 0.99 |
| Film Coating | 2.44 |

### Process

1. All the ingredients were sifted separately through appropriate sieves (100% should pass through sieve, e.g., 24 mesh).

2. The pre-sifted Compound of Formula 1, microcrystalline cellulose, croscarmellose sodium and colloidal silicon dioxide were dry mixed in a suitable blender.

3. The dry mixed ingredients of Step 2 in the blender were lubricated using approx . . . 50% of the sodium stearyl fumarate.

4. The blend of Step 3 was passed through a roller compactor to obtain compacted mass/ribbons.

5. The compacted mass/ribbons of Step 4 were passed through an appropriate mesh screen (e.g., 18 to 30 mesh) and the blend was passed through the required sieve (e.g., 18 to 30 mesh) to obtain granules.

6. The granules obtained from Step 5 were blended in a suitable blender and lubricated using the remaining sodium stearyl fumarate.

7. The blend of Step 6 was compressed into tablets.

8. The tablets of Step 7 were film coated.

### Example 4

| Ingredients | Qty/Tablet (mg) | | | | % w/w |
|---|---|---|---|---|---|
| Microcrystalline | 297.00 | 594.00 | 891.00 | 1485.00 | 49.50 |
| cellulose | | | | | |
| Crospovidone | 15.00 | 30.00 | 45.00 | 75.00 | 2.50 |
| Compound of Formula | 100.00 | 200.00 | 300.00 | 500.00 | 16.70 |
| I (e.g. Formula IA) | | | | | |
| Hypromellose | 70.00 | 140.00 | 210.00 | 350.00 | 11.66 |
| Sodium Lauryl Sulfate | 30.00 | 60.00 | 90.00 | 150.00 | 5.00 |
| Purified Water | q.s | q.s | q.s | q.s. | q.s |
| Microcrystalline | 50.00 | 100.00 | 150.00 | 250.00 | 8.30 |
| Cellulose | | | | | |
| Crospovidone | 30.00 | 60.00 | 90.00 | 150.00 | 5.00 |
| Colloidal Silicon | 3.00 | 6.00 | 9.00 | 15.00 | 0.50 |
| Dioxide | | | | | |
| Magnesium Stearate | 5.00 | 10.00 | 15.00 | 25.00 | 0.80 |
| Unit Weight (mg) | 600.00 | 1200.00 | 1800.00 | 3000.00 | 100.00 |

### Process:

1. All the ingredients were sifted separately through appropriate sieves.

2. The Compound of Formula I and the dry ingredients were mixed in a rapid mixer granulator (RMG).

3. Hydroxyl propyl methyl cellulose and sodium lauryl sulfate in purified water were added to the product of Step 2 and mixed until a homogenous dispersion (binder) was obtained.

4. The dry mixed ingredients of Step 2 were granulated in the RMG (rapid mixing granulator) with the binder of Step 3.

5. The wet granules of Step 4 were dried in a fluid bed dryer (FBD) until the required loss on drying (LOD) was achieved.

6. The dried granules were passed through an appropriate mesh (e.g., 18 to 30 mesh) and the oversized granules were milled using a multi-mill.

7. The granules of Step 6 were blended in a suitable blender.

8. The granules of Step 7 were blended with pre-sifted microcrystalline cellulose, crospovidone and colloidal silicon dioxide in a suitable blender.

9. The blend of Step 8 was lubricated with pre-sifted magnesium stearate in a blender.

10. The blend of Step 9 was compressed into tablets.

11. The tablets were packaged as required.

### Example 5

| Ingredients | Qty/Tablet (mg) | | | | % w/w |
|---|---|---|---|---|---|
| Microcrystalline Cellulose | 297.00 | 594.00 | 891.00 | 1485.00 | 49.50 |
| Crospovidone | 15.00 | 30.00 | 45.00 | 75.00 | 2.50 |
| Compound of Formula IA | 100.00 | 200.00 | 300.00 | 500.00 | 16.70 |
| Hypromellose | 100.00 | 200.00 | 300.00 | 500.00 | 16.70 |
| Methylene Chloride | q.s | q.s | q.s | q.s. | q.s |
| Microcrystalline Cellulose | 50.00 | 100.00 | 150.00 | 250.00 | 8.30 |
| Crospovidone | 30.00 | 60.00 | 90.00 | 150.00 | 5.00 |
| Colloidal Silicon Dioxide | 3.00 | 6.00 | 9.00 | 15.00 | 0.50 |
| Magnesium Stearate | 5.00 | 10.00 | 15.00 | 25.00 | 0.80 |
| Unit weight (mg) | 600.00 | 1200.00 | 1800.00 | 3000.00 | 100.00 |

### Process

1. All the ingredients were sifted separately through appropriate sieves (e.g., 24 to 40 mesh).

2. The dry ingredients [microcrystalline cellulose and crospovidone) were mixed in a RMG.

3. The Compound of Formula IA in methylene chloride was added and mixed until a clear solution formed. Hydroxyl propyl methyl cellulose in methylene chloride was then added and mixed until a homogenous dispersion (binder) was obtained.

4. The dry mixed ingredients of Step 2 were mixed in the RMG with the binder of Step 3.

5. The wet granules of Step 4 were dried in a FBD until the required LOD was achieved.

6. The dried granules of Step 5 were passed through an appropriate mesh (e.g., 18 to 30 mesh) and the oversized granules were milled using a multi-mill.

7. The granules of Step 6 were blended in a suitable blender.

8. The granules of Step 7 were blended with pre-sifted microcrystalline cellulose, crospovidone and colloidal silicon dioxide in a suitable blender.

9. The blend of Step 8 was blended with pre-sifted magnesium stearate in a blender.

10. The blend of Step 9 was compressed into tablets.

11. The tablets were packaged as required.

### Example 6

| Ingredients | Amount | | | | | |
|---|---|---|---|---|---|---|
| Compound of | 50.00 | 100.00 | 200.00 | 300.00 | 400.00 | 500.00 |
| Formula IA | mg | mg | mg | mg | mg | mg |
| 2- | 375 | 750 | 1500 | 2250 | 3000 | 3750 |
| Hydroxypropyl | mg | mg | mg | mg | mg | mg |
| Beta- | | | | | | |
| Cyclodextrin | | | | | | |
| Sterile Water | 7.5 | 15.00 | 30.00 | 45.00 | 60.00 | 75.00 |
| | ml | ml | ml | ml | ml | ml |

### Process

1. The 2-hydroxypropyl beta-cyclodextrin and Compound of Formula 1A were weighed.

2. Water for injection was added to the 2-hydroxypropyl beta-cyclodextrin to obtain a 5% w/v solution.

3. The 5% solution of 2-hydroxypropyl beta-cyclodextrin of Step 2 was added to the Compound of Formula 1A with stirring to obtain a suspension.

### Example 7

| Ingredients | Qty/Tablet (mg) | | | | % w/w |
|---|---|---|---|---|---|
| Pearlitol-Flash | 210.00 | 420.00 | 630.00 | 1050.00 | 35.00 |
| (D-Mannitol 80%- | | | | | |
| Dehydrated Extra | | | | | |
| White Maize Starch | | | | | |
| 20%) | | | | | |
| Crospovidone | 20.00 | 40.00 | 60.00 | 100.00 | 3.30 |
| 2-Hydroxypropyl | 20.00 | 40.00 | 60.00 | 100.00 | 3.30 |
| Beta-Cyclodextrin | | | | | |
| Compound of | 100.00 | 200.00 | 300.00 | 500.00 | 16.70 |
| Formula IA | | | | | |
| Hypromellose | 100.00 | 200.00 | 300.00 | 500.00 | 16.70 |
| Methylene Chloride | q.s | q.s. | q.s. | q.s. | q.s |
| Crospovidone | 15.00 | 30.00 | 45.00 | 75.00 | 2.50 |
| Sodium Starch | 15.00 | 30.00 | 45.00 | 75.00 | 2.50 |
| Glycolate | | | | | |
| Colloidal Silicon | 3.00 | 6.00 | 9.00 | 15.00 | 0.50 |
| Dioxide | | | | | |
| Pearlitol-Flash | 112.00 | 224.00 | 336.00 | 560.00 | 18.70 |
| (D-Mannitol 80%- | | | | | |
| Dehydrated Extra | | | | | |
| White Maize Starch | | | | | |
| 20%) | | | | | |
| Magnesium Stearate | 5.00 | 10.00 | 15.00 | 25.00 | 0.80 |
| Unit Weight (mg) | 600.00 | 1200.00 | 1800.00 | 3000.00 | 100.00 |

### Process

1. All the ingredients were sifted separately through appropriate sieves (e.g., 24 to 40 mesh).

2. The dry ingredients (Pearlitol-Flash, crospovidone, and 2-hydroxypropyl beta-cyclodextrin) were mixed in a RMG.

3. The Compound of Formula IA in methylene chloride was added and mixed until a clear solution formed. Hydroxyl propyl methyl cellulose in methylene chloride was then added and mixed until a homogenous dispersion (binder) was obtained.

4. The dry mixed ingredients of Step 2 were granulated in the RMG with the binder of Step 3.

5. The wet granules of Step 4 were dried in a FBD until the required LOD was achieved.

6. The dried granules of Step 8 were passed through an appropriate mesh (e.g., 18 to 30 mesh) and the oversized granules were milled using a multi-mill.

7. The granules of Step 6 were blended in a suitable blender.

8. The granules of Step 7 were blended with pre-sifted sodium starch glycolate, crospovidone, Pearlitol Flash and colloidal silicon dioxide in a suitable blender

9. The blend of Step 8 was lubricated with pre-sifted magnesium stearate in a blender.

10. The blend of Step 9 was compressed into tablets.

11. The tablets were packaged as required.

### Example 8

| Ingredients | Qty/Tablet (mg) | | | | % w/w |
|---|---|---|---|---|---|
| F-Melt Type C | 285.00 | 570.00 | 855.00 | 1425.00 | 47.5 |
| (D-Mannitol, Xylitol, | | | | | |
| Microcrystalline | | | | | |
| Cellulose, | | | | | |
| Crospovidone, | | | | | |
| Fujicalin) | | | | | |
| Crospovidone | 15.00 | 30.00 | 45.00 | 75.00 | 2.50 |
| Compound of | 100.00 | 200.00 | 300.00 | 500.00 | 16.70 |
| Formula IA | | | | | |
| Hypromellose | 100.00 | 200.00 | 300.00 | 500.00 | 16.70 |
| Methylene Chloride | q.s | q.s | q.s | q.s. | q.s |
| Crospovidone | 30.00 | 60.00 | 90.00 | 150.00 | 5.00 |
| Colloidal Silicon | 3.00 | 6.00 | 9.00 | 15.00 | 0.50 |
| dioxide | | | | | |
| F-Melt Type C | 50.00 | 100.00 | 150.00 | 250.00 | 8.30 |
| (D-Mannitol, Xylitol, | | | | | |
| Crospovidone, | | | | | |
| Microcrystalline | | | | | |
| cellulose, Fujicalin) | | | | | |
| Aspartame | 6.00 | 12.00 | 18.00 | 30.00 | 1.00 |
| Flavour Strawberry | 6.00 | 12.00 | 18.00 | 30.00 | 1.00 |
| Magnesium Stearate | 5.00 | 10.00 | 15.00 | 25.00 | 0.80 |
| Unit weight | 600.00 | 1200.00 | 1800.00 | 3000.00 | 100.00 |

### Process

1. All the ingredients were sieved separately through appropriate sieves (e.g., 24 to 40 mesh).

2. The dry ingredients (F-melt and crospovidone) were mixed in a RMG.

3. The Compound of Formula IA in methylene chloride was added and mixed until a clear solution formed. Hydroxyl propyl methyl cellulose in methylene chloride and mixed until a homogenous dispersion (binder) was obtained.

4. The dry mixed ingredients of Step 2 were granulated in the RMG with the binder of Step 3.

5. The wet granules of Step 4 were dried in a FBD until the required LOD was achieved.

6. The dried granules of Step 5 were passed through an appropriate mesh and the oversized granules were milled using a multi-mill.

7 The granules of Step 6 were blended in a suitable blender.

8. The granules of Step 7 were blended with pre-sifted F-melt, crospovidone, aspartame, strawberry flavour and colloidal silicon dioxide in a suitable blender.

9 The blend of Step 8 was blended with pre-sifted magnesium stearate in a blender.

10. The blend of Step 9 was compressed into tablets.

11. The tablets were packaged as required.

### Example 8A

| | Formulation 8A |
|---|---|
| Ingredients | Unit Dose |
| | (mg/tablet) |

Dry Mix

| | |
|---|---|
| F-Melt Type C | 510.00 |
| (D-Mannitol, Xylitol, | |
| Microcrystalline | |
| Cellulose, | |
| Crospovidone, | |
| Fujicalin) | |
| Aeroperl 300 | 20.00 |
| Polyplasdone XL 10 | 31.00 |
| Gelucire 59/14 | 6.00 |
| (Gattefose) | |

Binder

| | |
|---|---|
| Compound of | 500.00 |
| Formula IA | |
| PVPK 29/32 | 18.00 |
| MDC | 300 grams |
| (methylene dichloride) | |

Prelubrication

| | |
|---|---|
| Polyplasdone XL 10 | 30.00 |

Lubrication

| | |
|---|---|
| Magnesium Stearate | 5.00 |
| (Ligamed MF-2-V) | |

### Process

### 1. Sifting

The F Melt (D-mannitol, Xylitol, MCC, Crospovidone Fujicalin), Aeroperl 300, Polyplasdone XL 10, Gelucire 59/14 (Gattefose) were sifted through a 40 #mesh and mixed.

### 2. Preparation of Binder

300 grams of methylene dichloride was split into three parts (200 g, 50 g, 50 g) and added to different glass beakers.

The compound of Formula IA was dissolved in 200 g of MDC by slowly adding it with continuous stirring until a clear slightly brown solution formed.

The PVPK 29/32 was dissolved in 50 g MDC is a separate beaker to give a clear solution, which was then added to the compound of Formula IA solution with stirring to give alighr brown solution (binder/API Solution).

### 3. Addition of Binder

The binder/API solution was slowly added to the sifted and premixed ingredients of Step 1 with continuous mixing. The rate of addition was slow and consistent to avoid the formation of lumps. After complete addition of the binder/API solution, the beaker was rinsed with 50 g of MDC and the rinsings were added and mixed to form a uniform wet mass.

### 4. Drying

The wet mass was dried in a fluidized bed drier at 50° C. for 20 minutes at an air flow of 20 to 25 cfm. The LOD obtained was 1.0 to 1.5.

### 5. Blending and Lubrication

The dried granules were passed through 30 #mesh and mixed with polyplasdone XL 10 for 2 to 3 minutes. Pre sifted (60 #mesh) magnesium stearate was added to the and blended for 2 minutes.

### 6. Compression

The blend was compressed with the following parameters.

Punch details: Oval shaped 19.00×9.90 mm punches

Average wt. of the tablet: 1120 mg

DT: 2 to 3 Minutes

Hardness: 12 kg

Thickness: 6.80 to 6.90 mm

### Dissolution Testing

Dissolution testing was conducted for Formulation 8A under the following conditions:

### Dissolution Medium

1. 0.01N HCl+1% SLS medium.
Number of tablets: 12
Sampling Time intervals 5 min, 10 min, 15 min, 20 min, 30 min, 45 min and 60 minutes.

### Apparatus: USP II (Paddle)

### Volume: 900 ml

### Speed: 100 rpm

### Temperature: 37.0±0.5° C.

### Results

| Time | | | | | | | |
|---|---|---|---|---|---|---|---|
| (Minutes) | 5 | 10 | 15 | 20 | 30 | 45 | 60 |
| % | 90.4 | 89.7 | 91.1 | 87.9 | 88.5 | 93.0 | 93.7 |
| Dissolution | | | | | | | |
| (Average) | | | | | | | |

### Example 9

| | Units Dose (Mg/Tablet) | | |
|---|---|---|---|
| Ingredient | Formulation 9A | Formulation 9B | Formulation 9C |

A-Dry Mixing

| | | | |
|---|---|---|---|
| F-Melt Type C (D- | 140.00 | 143.00 | 147.00 |
| Mannitol, | | | |
| Xylitol, MCC, | | | |
| Crospovidone, | | | |
| Fujicalin) | | | |
| Aeroperl 300 | 8.00 | 10.00 | 8.00 |
| Polyplasdone XL 10 | 19.00 | 20.00 | 20.00 |
| Gelucire 59/14 | 5.00 | 5.00 | 5.00 |
| (Gattefose) | | | |

B-Binder

| | | | |
|---|---|---|---|
| Compound of | 100.00 | 100.00 | 100.00 |
| Formula IA | | | |
| PVPK 29/32 | 6.00 | 10.00 | 8.00 |
| Methylene Chloride | q.s. | q.s. | q.s. |

C-Pre-Lubrication

| | | | |
|---|---|---|---|
| Polyplasdone XL 10 | 20.00 | 20.00 | 20.00 |

D-Lubrication

| | | | |
|---|---|---|---|
| Magnesium Stearate | 2.00 | 2.00 | 2.00 |

* - F-Melt is available from Fuji Chemical Industries USA, Inc. of Burlington Township, N.J., Aeroperl 300 is colloidal silicon dioxide, Polyplasdone XL 10 (available from Ashland of Rotterdam, Netherlands) is crospovidone, Gelucire 59/14 (available from Gattefossé of Saint-Priest, France) is a mixture of lauroyl polyoxy1-32 glycerides and PEG 6000.

### Process

### Step 1—Sifting and Dry Mixing

The F Melt (D-mannitol, Xylitol, MCC, Crospovidone Fujicalin), Aeroperl 300, Polyplasdone XL 10 and Gelucire 59/14 (Gattefose) were sieved (#40 sieve) and mixed for 5 minutes.

### Step 2—Binder Preparation

PVP 29/32 was added to 100 grams methylene chloride with stirring to give a clear solution. The Compound of Formula IA was added slowly with stirring to give a light brown solution.

### Step 3—Binder Addition

The binder preparation of Step 2 was added to the dry mixed ingredients of Step 1 with continuous mixing. The wet mass was allowed to mix uniformly before the addition of further increments of the binder preparation. The binder preparation was added completely and mixed thoroughly. The binder preparation container was rinsed with 50 grams of methylene chloride which was slowly added to the wet mass with continuous mixing.

### Step 4—Drying

The wet mass of Step 4 was dried using a FBD. Initial drying was performed at 40° C. (inlet temperature) for 20 minutes. The semidried mass was then passed through a sieve (#20 sieve) and again dried in the FBD at 50-55° C. for another 20-30 minutes. The final LOD of the granules was 2.0 to 2.5%. (IR moisture balance at 105° C.)

### Step 5—Sizing

The dried granules of Step 4 were passed through a #30 sieve. The sized granules were transferred to double lined poly bag (2.0 kg capacity).

### Step 6—Blending

Polyplasdone XL 10 was passed through a #40 sieve, added to the dried granules of Step 5 and mixed for 5 minutes. Magnesium stearate was passed through a #60 sieve number, added to the dried granules of Step 5 and mixed for 5 minutes.

### Step 7—Compression

The blend was compressed with the following parameters.

| | Formulation 9A | Formulation 9B | Formulation 9C |
|---|---|---|---|
| Average Tablet | 300 | 310 | 310 |
| Weight (mg) | | | |
| Disintegration | 30-40 | 50-60 | 50-60 |
| Time (DT) | | | |
| (seconds) | | | |
| Tablet Hardness | 4-5 | 4-5 | 4-5 |
| (Kg) | | | |
| LOD (%) | 1.5-2.0 | 1.5-2.0 | 1.5-2.0 |

### Dissolution Testing

Dissolution testing was conducted for Formulations 9A, 9B and 9C under the following conditions:

### Formulations 9A and 9B

### Dissolution MEDIUM:

1. Simulated gastric fluid (without Pepsin): 2.0 gm of NaCl+7.0 ml of HCl, Makeup to 1000 ml with purified water, pH 1.2

2. 0.01N HCl+1% SLS (sodium lauryl sulfate) medium.

### Formulation 9C

### Dissolution Medium:

1. 0.01N HCl+1% SLS medium.

2. 0.1N HCl+1% SLS medium

3. pH 4.5 Acetate+1% SLS medium

4. pH 6.8 Phosphate+1% SLS medium

### Test Conditions For All Media:

Number of tablets: 12

Sampling Time Intervals: 30, 45 and 60 minutes

Apparatus: USP II (Paddle)

Volume: 900 mL

Speed: 100 rpm

Temperature: 37.0±0.5° C.

Average wt. of the tablet for Formulation 9A: 300 mg

Average wt. of the tablet for Formulation 9B: 310 mg

Average wt. of the tablet for Formulation 9C: 310 mg

### Results

### Formulations 9A and 9B

| % | Medium 1 | | | Medium 2 | | |
|---|---|---|---|---|---|---|
| Dissolution | 30 Mins | 45 Mins | 60 Mins | 30 Mins | 45 Mins | 60 Mins |
| Formulation | | | | | | |
| 9A | | | | | | |
| Average (%) | 68.45 | 71.2 | 63.8 | 97.30 | 95.7 | 94.9 |
| Formulation | | | | | | |
| 9B | | | | | | |
| Average (%) | 52.70 | 37.90 | 30.00 | 95.45 | 94.40 | 94.10 |

### Formulation 9C

| % | Medium 1 | | | Medium 2 | | |
|---|---|---|---|---|---|---|
| Dissolution | 30 Mins | 45 Mins | 60 Mins | 30 Mins | 45 Mins | 60 Mins |
| Formulation | | | | | | |
| 9C | | | | | | |
| Average (%) | 95.37 | 93.10 | 92.50 | 94.88 | 94.30 | 93.90 |
| % | Medium 3 | | | Medium 4 | | |
| Dissolution | 30 Mins | 45 Mins | 60 Mins | 30 Mins | 45 Mins | 60 Mins |
| Formulation | | | | | | |
| 9C | | | | | | |
| Average (%) | 92.43 | 92.30 | 91.55 | 93.93 | 93.70 | 93.50 |

### Stability Testing

Stability and dissolution testing was conducted for Formulations 9A, 9B and 9C at 40° C. and 75% Relative Humidity (RH). The results are shown below.

| Formulation 9A | Initial | 1.5 Months | 3 Months | 6 Months |
|---|---|---|---|---|
| Assay | 96.2% | 97.1% | 97.5% | 96.2% |
| Unknown Impurity | 0.02% | 0.02% | 0.01% | 0.02% |
| (RT 0.77) | | | | |
| Unknown Impurity | 0.02% | 0.02% | 0.01% | ND |
| (RT 1.06) | | | | |
| Highest Unknown | 0.02% | 0.02% | 0.01% | 0.02% |
| Impurity | | | | |

% Dissolution¹

| Time (mins) | Avg (min-max) | | Avg (min-max) | | Avg (min-max) | | Avg (min-max) | |
|---|---|---|---|---|---|---|---|---|
| 5 | 90 | (88-93) | 91 | (90-91) | 92 | (91-93) | 87 | (85-88) |
| 10 | 92 | (91-93) | 94 | (93-96) | 95 | (94-96) | 95 | (94-96) |
| 15 | 93 | (92-94) | 94 | (93-95) | 96 | (93-97) | 96 | (94-97) |
| 20 | 93 | (92-94) | 94 | (91-96) | 95 | (94-96) | 96 | (94-97) |
| 30 | 93 | (91-94) | 93 | (91-95) | 95 | (93-96) | 95 | (94-97) |
| 45 | 92 | (90-93) | 93 | (91-95) | 95 | (94-96) | 95 | (94-96) |
| 60 | 92 | (90-93) | 94 | (93-96) | 96 | (94-96) | 95 | (93-97) |
| 90 | 93 | (91-95) | 94 | (93-96) | 96 | (94-96) | 95 | (93-97) |

¹0.01 N HCl + 1% SLS, 900 mL, 100 RPM, USP II (Paddle)

| Formulation 9B | Initial | 1.5 Months | 3 Months | 6 Months |
|---|---|---|---|---|
| Assay | 96.3% | 95.8% | 96.3% | 95.7% |
| Unknown Impurity | 0.01% | ND | ND | ND |
| (RT 0.41) | | | | |
| Unknown Impurity | 0.02% | 0.02% | 0.02% | 0.01% |
| (RT 0.77) | | | | |
| Unknown Impurity | 0.02% | 0.02% | 0.02% | ND |
| (RT 1.06) | | | | |
| Highest Unknown | 0.02% | 0.02% | 0.02% | 0.01% |
| Impurity | | | | |

% Dissolution¹

| Time (mins) | Avg (min-max) | | Avg (min-max) | | Avg (min-max) | | Avg (min-max) | |
|---|---|---|---|---|---|---|---|---|
| 5 | 90 | *88-93) | 20 | (14-24) | 8 | (6-10) | 4 | (4-5) |
| 10 | 92 | (90-94) | 53 | (40-61) | 27 | (24-30) | 16 | (14-18) |
| 15 | 92 | (90-94) | 81 | (67-90) | 48 | (45-54) | 30 | (28-43) |
| 20 | 92 | (89-97) | 92 | (89-95) | 70 | (64-79) | 45 | (42-50) |
| 30 | 92 | (90-95) | 96 | (93-100) | 91 | (89-93) | 77 | (69-81) |
| 45 | 92 | (89-45) | 96 | (93-100) | 94 | (91-98) | 92 | (90-93) |
| 60 | 92 | (89-96) | 96 | (92-99) | 95 | (93-97) | 93 | (91-94) |
| 90 | 94 | (92-97) | 95 | (92-98) | 96 | (93-99) | 92 | (91-93) |

¹0.01 N HCl + 1% SLS, 900 mL, 100 RPM, USP II (Paddle)

| | | | | |
|---|---|---|---|---|
| Formulation 9C | Initial | 1.5 Months | 3 Months | 6 Months |

Assay

| | | | | |
|---|---|---|---|---|
| Unknown Impurity | 0.01% | ND | ND | ND |
| (RT 0.17) | | | | |
| Unknown Impurity | 0.02% | 0.01% | 0.02% | 0.02% |
| (RT 0.77) | | | | |
| Unknown Impurity | 0.02% | 0.02% | 0.02% | 0.02% |
| (RT 1.06) | | | | |
| Highest Unknown | 0.02% | 0.02% | 0.02% | 0.02% |
| Impurity | | | | |

% Dissolution¹

| Time (mins) | Avg (min-max) | | Avg (min-max) | | Avg (min-max) | | Avg (min-max) | |
|---|---|---|---|---|---|---|---|---|
| 5 | 89 | (85-91) | 50 | (44-56) | 26 | (19-29) | 12 | (9-18) |
| 10 | 93 | (92-94) | 89 | (88-90) | 60 | (52-67) | 37 | (32-46) |
| 15 | 93 | (92-94) | 92 | (91-93) | 80 | (56-88) | 59 | (54-68) |
| 20 | 93 | (92-94) | 92 | (91-94) | 91 | (89-91) | 76 | (71-84) |
| 30 | 93 | (92-94) | 93 | (91-95) | 92 | (90-94) | 91 | (89-92) |
| 45 | 93 | (92-96) | 93 | (91-95) | 92 | (90-94) | 92 | (91-94) |
| 60 | 93 | (92-96) | 93 | (92-95) | 92 | (90-94) | 92 | (91-94) |
| 90 | 94 | (92-97) | 93 | (91-95) | 92 | (91-94) | 92 | (91-94) |

¹0.01 N HCl + 1% SLS, 900 mL, 100 RPM, USP II (Paddle)

### Example 10

| | Unit Dose (Mg/Tablet) | |
|---|---|---|
| Ingredient | Formulation 10A | Formulation 10B |

A-Dry Mixing

| | | |
|---|---|---|
| F Melt (D-Mannitol, | 240.00 | 259.00 |
| Xylitol, MCC, | | |
| Crospovidone Fujicalin) | | |
| Aeroperl 300 | 8.00 | 8.00 |
| Polyplasdone XL 10 | 19.00 | 19.00 |
| Gelucire 59/14 | 5.00 | 4.00 |
| (Gattefose) | | |

B-Binder

| | | |
|---|---|---|
| Compound of Formula | 200.00 | 200.00 |
| IA | | |
| PVPK 29/32 | 12.00 | 8.00 |
| Methylene Chloride | q.s. | q.s. |

C-Pre-Lubrication

| | | |
|---|---|---|
| Polyplasdone XL 10 | 25.00 | 20.00 |

D-Lubrication

| | | |
|---|---|---|
| Magnesium Stearate | 3.00 | 2.00 |

### Process

### Step 1—Sifting and Dry Mixing

The F Melt (D-mannitol, Xylitol, MCC, Crospovidone Fujicalin), Aeroperl 300, Polyplasdone XL 10 and Gelucire 59/14 (Gattefose) were sieved (#40 sieve) and mixed for 5 minutes.

### Step 2—Binder Preparation

PVP 29/32 was added to 150 grams methylene chloride with stirring to give a clear solution. The Compound of Formula IA was added slowly with stirring to give a light brown solution.

### Step 3—Binder Addition

The binder preparation of Step 2 was added to the dry mixed ingredients of Step 1 with continuous mixing. The wet mass was allowed to mix uniformly before the addition of further increments of the binder preparation. The binder preparation was added completely and mixed thoroughly. The binder preparation container was rinsed with 50 grams of methylene chloride which was slowly added to the wet mass with continuous mixing.

### Step 4—Drying

The wet mass of Step 4 was dried using a FBD. Initial drying was performed at 40° C. (inlet temperature) for 20 minutes. The semidried mass was then passed through a sieve (#20 sieve) and again dried in the FBD at 50-55° C. for another 20-0 minutes. The final LOD of the granules was 2.0 to 2.5%. (IR moisture balance at 105° C.)

### Step 5—Sizing

The dried granules of Step 4 were passed through a #30 sieve. The sized granules were transferred to double lined poly bag (2.0 kg capacity).

### Step 6—Blending

Polyplasdone XL 10 was passed through a #40 sieve, added to the dried granules of Step 5 and mixed for 5 minutes. Magnesium stearate was passed through a #60 sieve number, added to the dried granules of Step 5 and mixed for 5 minutes.

### Step 7—Compression

The blend was compressed with the following parameters.

| | Formulation 10A | Formulation 10B |
|---|---|---|
| Average Tablet | 520 | 520 |
| Weight (mg) | | |
| Dissolution Time | 55-60 | 20 |
| (seconds) | | |
| Tablet Hardness Kg | 4-5 | 4-5 |
| LOD (%) | 1.5-2.0 | 2.5-3.0 |

### Dissolution Testing

Dissolution testing was conducted for Formulations 10A and 10B under the following conditions:

### Formulation 10A

### Dissolution Medium:

1. 0.01N HCl+1% SLS medium.

2. 0.1N HCl+1% SLS medium

3. pH 4.5 Acetate+1% SLS medium

4. pH 6.8 Phosphate+1% SLS medium

### Formulations 10B

### Dissolution Medium:

1. 0.01N HCl+1% SLS medium.

### Test Conditions For All Media:

Number of tablets: 12

Sampling Time Intervals: 30, 45 and 60 minutes (Formulation 10A)

Sampling Time Intervals: 5, 10, 15, 20, 30, 45 and 60 minutes (Formulation 10B)

Apparatus: USP II (Paddle)

Volume: 900 mL

Speed: 100 rpm

Temperature: 37.0±0.5° C.

Average wt. of the tablet for Formulation 10A: 520 mg

Average wt. of the tablet for Formulation 10B: 520 mg

### Formulation 10A

| % | Medium 1 | | | Medium 2 | | |
|---|---|---|---|---|---|---|
| Dissolution | 30 Mins | 45 Mins | 60 Mins | 30 Mins | 45 Mins | 60 Mins |
| Formulation | | | | | | |
| 10A | | | | | | |
| Average (%) | 96.90 | 96.80 | 96.50 | 89.11 | 89.50 | 90.10 |
| % | Medium 3 | | | Medium 4 | | |
| Dissolution | 30 Mins | 45 Mins | 60 Mins | 30 Mins | 45 Mins | 60 Mins |
| Formulation | | | | | | |
| 10A | | | | | | |
| Average (%) | 95.11 | 94.60 | 94.10 | 93.26 | 93.70 | 93.60 |

### Formulation 10B

| | Medium 1 | | | | | | |
|---|---|---|---|---|---|---|---|
| % | 5 | 10 | 15 | 20 | 30 | 45 | 60 |
| Dissolution | Mins | Mins | Mins | Mins | Mins | Mins | Mins |
| Formulation | | | | | | | |
| 10B | | | | | | | |
| Average (%) | 98.1 | 99.5 | 100.5 | 101.6 | 102.7 | 103.6 | 105.3 |

### Stability Testing

Stability and dissolution testing was conducted for Formulation 10A at 40° C. and 75% Relative Humidity (RH). The results are shown below.

| Formulation 10A | Initial | 1.5 Months | 3 Months | 6 Months |
|---|---|---|---|---|
| Assay | 96.8% | 95.5% | 94.1% | 96.3% |
| Unknown Impurity | 0.02% | 0.01% | 0.02% | 0.03% |
| (RT 0.77) | | | | |
| Unknown Impurity | 0.02% | 0.02% | 0.02% | ND |
| (RT 1.06) | | | | |
| Highest Unknown | 0.02% | 0.02% | 0.02% | 0.03% |
| Impurity | | | | |

% Dissolution¹

| Time (mins) | Avg (min-max) | | Avg (min-max) | | Avg (min-max) | | Avg (min-max) | |
|---|---|---|---|---|---|---|---|---|
| 5 | 88 | (83-90) | 22 | (16-33) | 3 | (2-5) | 2 | (1-2) |
| 10 | 88 | (84-90) | 50 | (36-73) | 10 | (7-16) | 3 | (3-4) |
| 15 | 88 | (87-89) | 72 | (61-91) | 18 | (13-27) | 6 | (5-6) |
| 20 | 88 | (86-88) | 90 | *84-94) | 29 | (21-41) | 9 | (8-10) |
| 30 | 88 | (86-89) | 94 | (93-94) | 50 | (41-69) | 15 | (14-18) |
| 45 | 88 | (87-89) | 94 | (93-94) | 80 | (72-92) | 36 | (23-30) |
| 60 | 88 | (88-89) | 94 | (94-94) | 91 | (89-93) | 39 | (34-46) |
| 90 | 89 | (88-89) | 94 | (94-95) | 93 | (92-95) | 75 | (70-79) |

¹0.01 N HCl + 1% SLS, 900 mL, 100 RPM, USP II (Paddle)

### Example 11

| | |
|---|---|
| | Unit Dose |
| | (Mg/Tablet) |
| Ingredient | Formulation 11A |

A-Dry Mixing

| | |
|---|---|
| F Melt (D-Mannitol, | 314.00 |
| Xylitol, MCC, | |
| Crospovidone Fujicalin | |
| Aeroperl 300 | 20.00 |
| Polyplasdone XL 10 | 30.00 |
| Gelucire 59/14 | 6.00 |
| (Gattefose) | |

B-Binder

| | |
|---|---|
| Compound of Formula | 300.00 |
| IA | |
| PVPK 29/32 | 18.00 |
| Methylene Chloride | 250 g |

C-Pre-Lubrication

| | |
|---|---|
| Polyplasdone XL 10 | 29.00 |

D-Lubrication

| | |
|---|---|
| Magnesium Stearate | 3.00 |

### Process

### Step 1—Sifting of Ingredients

F Melt (D-mannitol, Xylitol, MCC, Crospovidone Fujicalin), Aeroperl 300, Polyplasdone XL 10, Gelucire 59/14 (Gattefose) were sieved (#40 sieve) and mixed for 2-3 minutes.

### Step 2—Preparation of Binder

250 g of methylene chloride was divided into three parts (150 g, 50 g, 50 g) and added to different in different glass beakers.

In one flask, the Compound of Formula IA was dissolved in 150 g of methylene chloride by slowly adding the Compound of Formula IA and stirring continuously to give a clear slightly brown solution.

In another flask with 50 grams of PVPK 29/32 was dissolved in 50 g methylene chloride to give a clear solution.

The PVPK 29/32 solution was added to the solution of the Compound of Formula IA slowly with stirring to give a clear slightly brown solution (Binder/API Solution)

### Step 3—Addition of Binder

The Binder/API solution of Step 2 was slowly added to the sifted and premixed ingredients of Step 1 with continuous mixing. The rate of addition was slow and consistent to avoid the formation of lumps. After complete addition of the Binder/API solution, the flask was rinsed with 50 g of methylene chloride and add the rinsings added to the remaining contents and mixed thoroughly to form a uniform wet mass.

### Step 4—Drying The Wet Mass

The wet mass of Step 3 was dried in a FBD at 50° C. for 20 minutes (air flow of 20 to 25 cfm). The LOD was 1.5 to 2.0%.

### Step 5—Blending and Lubrication

The dried granules were passed through 30 #mesh and mixed with Polyplasdone XL 10 in a poly bag for 2 to 3 minutes. Pre sifted (60 #) Magnesium Stearate was added to the above mixture and blended for 2 minutes in a polybag.

### Step 6—Compression

The blend was compressed with the following parameters.

| | Formulation 11A |
|---|---|
| Punch Details | Oval Shaped |
| | 16.40 × 8.20 mm |
| Average Tablet | 720 |
| Weight (mg) | |
| Dissolution Time | 20-30 |
| (seconds) | |
| Tablet Hardness (kg) | 4-5 |
| Thickness (mm | 5.5 |

### Dissolution Testing

Dissolution testing was conducted for Formulation 11A under the following conditions:

### Formulation 11A

### Dissolution Medium:

1. 0.01N HCl+1% SLS medium.

2. 0.1N HCl+1% SLS medium

3. pH 4.5 Acetate+1% SLS medium

4. pH 6.8 Phosphate+1% SLS medium

### Test Conditions For All Media:

Number of tablets: 12

Sampling Time Intervals: 30, 45 and 60 minutes

Apparatus: USP II (Paddle)

Volume: 900 mL

Speed: 100 rpm

Temperature: 37.0±0.5° C.

Average wt. of the tablet for Formulation 11A: 720 mg

### Formulation 11A

| % | Medium 1 | | | Medium 2 | | |
|---|---|---|---|---|---|---|
| Dissolution | 30 Mins | 45 Mins | 60 Mins | 30 Mins | 45 Mins | 60 Mins |
| Formulation | | | | | | |
| 11A | | | | | | |
| Average (%) | 97.57 | 97.20 | 96.40 | 83.58 | 84.30 | 84.80 |
| % | Medium 3 | | | Medium 4 | | |
| Dissolution | 30 Mins | 45 Mins | 60 Mins | 30 Mins | 45 Mins | 60 Mins |
| Formulation | | | | | | |
| 11A | | | | | | |
| Average (%) | 96.08 | 96.50 | 96.20 | 96.69 | 101.00 | 98.10 |

### Stability Testing

Stability and dissolution testing was conducted for Formulation 10A at 40° C. and 75% Relative Humidity (RH). The results are shown below.

| Formulation 10A | Initial | 1.5 Months | 3 Months | 6 Months |
|---|---|---|---|---|
| Assay | 96.8% | 95.5% | 94.1% | 96.3% |
| Unknown Impurity | ND | 0.00% | 0.02% | 0.01% |
| (RT 0.27) | | | | |
| Unknown Impurity | 0.00% | 0.00% | 0.03% | 0.00% |
| (RT 0.32) | | | | |
| Unknown Impurity | 0.00% | 0.00% | 0.03% | 0.00% |
| (RT 0.36) | | | | |
| Unknown Impurity | 0.00% | 0.00% | 0.02% | 0.00% |
| (RT 0.49) | | | | |
| Unknown Impurity | 0.02% | 0.02% | 0.02% | 0.02% |
| (RT 0.76) | | | | |
| Unknown Impurity | ND | 0.00% | 0.02% | 0.02% |
| (RT 1.06) | | | | |
| Unknown Impurity | 0.00% | 0.00% | 0.03% | 0.00% |
| (RT 2.05) | | | | |
| Highest Unknown | 0.02% | 0.02% | 0.03% | 0.02% |
| Impurity | | | | |

% Dissolution¹

| Time (mins) | Avg (min-max) | | Avg (min-max) | | Avg (min-max) | | Avg (min-max) | |
|---|---|---|---|---|---|---|---|---|
| 5 | 88 | (87-89) | 87 | (86-88) | 81 | (79-82) | 57 | (55-61) |
| 10 | 94 | (93-95) | 96 | (94-96) | 93 | (93-94) | 83 | (81-85) |
| 15 | 95 | (94-95) | 97 | (95-97) | 95 | (95-96) | 92 | (91-93) |
| 20 | 93 | (93-95) | 97 | (95-97) | 96 | (95-97) | 96 | (95-97) |
| 30 | 95 | (94-95) | 96 | (94-98) | 96 | (95-97) | 98 | (97-99) |
| 45 | 95 | (94-95) | 97 | (95-97) | 96 | (95-97) | 99 | (98-100) |
| 60 | 95 | (94-96) | 97 | (95-97) | 96 | (95-97) | 98 | (98-99) |
| 90 | 95 | (94-96) | 97 | (95-97) | 97 | (96-98) | 99 | (98-100) |

¹0.01 N HCl + 1% SLS, 900 mL, 100 RPM, USP II (Paddle)

### Example 12

| | Dose | | | | | |
|---|---|---|---|---|---|---|
| | 50 | 100 | 200 | 300 | 400 | 500 |
| Ingredient | mg | mg | mg | mg | mg | mg |
| Compound of | 50.00 | 100.00 | 200.00 | 300.00 | 400.00 | 500.00 |
| Formula IA | | | | | | |
| (2- | 375 | 750 | 1500 | 2250 | 3000 | 3750 |
| Hydroxylpropyl) | mg | mg | mg | mg | mg | mg |
| Beta- | | | | | | |
| Cyclodextrin | | | | | | |
| Sterile Water | 7.50 | 15.0 | 30.0 | 45.0 | 60.0 | 75.0 |
| | ml | ml | ml | ml | ml | ml |
| Dose Strength | 6.66 | 6.66 | 6.66 | 6.66 | 6.66 | 6.66 |
| | mg/ml | mg/ml | mg/ml | mg/ml | mg/ml | mg/ml |

### Process

### Step 1—Dispensing

Required amounts of the Compound of Formula IA and (2-hydroxylpropyl) beta-cyclodextrin (HPPCD) were weighed.

### Step 2—Mixing and Sonication

Sterile water for injection was added to the (2-hydroxylpropyl) beta-cyclodextrin to obtain a 5% solution w/v. Half the volume of the 5% HPPCD solution was added to the Compound of Formula I with mixing. The resulting material was sonicated for 15 minutes. The remaining half volume of the 5% HPPCD solution was then added and the resulting suspension sonicated for a further 15 minutes

### Step 3—Stirring

The suspension prepared in Step 2 was then stirred at 900-1200 RPM for 60 minutes to afford a white to off-white suspension.

Maintain a temperature of 20-25° C. during all process steps.

Store final suspension at 20-25° C. for not more than 24 hours.

### Stability Studies

### Formulation 12A

| | 0 Hrs | | | | | |
|---|---|---|---|---|---|---|
| Time | (Initial) | 6 Hrs | 12 Hrs | 18 Hrs | 24 Hrs | Comments |
| | 25 ± 2° C./60 ± 5% RH − 9 Months | | | | | |
| Highest | 0.010 | 0.008 | 0.011 | 0.010 | 0.009 | White to |
| Unknown | | | | | | Off-White |
| Impurity (%) | | | | | | Suspension |
| Total | 0.024 | 0.028 | 0.025 | 0.019 | 0.017 | |
| Impurities | | | | | | |
| (%) | | | | | | |
| Assay (% of | 98.1 | 98.0 | 98.2 | 98.1 | 99.5 | |
| 6.66 mg/ml) | | | | | | |
| | 30 ± 2° C./65 ± 5% RH − 9 Months | | | | | |
| Highest | 0.013 | 0.011 | 0.015 | 0.011 | 0.012 | White to |
| Unknown | | | | | | Off-White |
| Impurity (%) | | | | | | Suspension |
| Total | 0.026 | 0.031 | 0.037 | 0.033 | 0.025 | |
| Impurities | | | | | | |
| (%) | | | | | | |
| Assay (% of | 100.1 | 100.6 | 100.6 | 100.3 | 100.3 | |
| 6.66 mg/ml) | | | | | | |
| | 30 ± 2° C./75 ± 5% RH − 6 Months | | | | | |
| Highest | 0.007 | 0.007 | 0.008 | 0.009 | 0.006 | White to |
| Unknown | | | | | | Off-White |
| Impurity (%) | | | | | | Suspension |
| Total | 0.016 | 0.012 | 0.013 | 0.014 | 0.010 | |
| Impurities | | | | | | |
| (%) | | | | | | |
| Assay (% of | 98.5 | 98.3 | 99.5 | 99.9 | 99.5 | |
| 6.66 mg/ml) | | | | | | |

Mean Particle Size Parameters: D₁₀ = 3.29 μm, D₅₀ = 13.2 μm, D₉₀ = 48.4 μm

### Formulation 12B

| | 0 Hrs | | | | | |
|---|---|---|---|---|---|---|
| Time | (Initial) | 6 Hrs | 12 Hrs | 18 Hrs | 24 Hrs | Comments |
| | 25 ± 2° C./60 ± 5% RH − 9 Months | | | | | |
| Highest | 0.010 | 0.008 | 0.009 | 0.011 | 0.008 | White to |
| Unknown | | | | | | Off-White |
| Impurity (%) | | | | | | Suspension |
| Total | 0.016 | 0.021 | 0.023 | 0.018 | 0.014 | |
| Impurities | | | | | | |
| (%) | | | | | | |
| Assay (% of | 100.2 | 100.3 | 100.2 | 100.3 | 99.4 | |
| 6.66 mg/ml) | | | | | | |
| | 30 ± 2° C./65 ± 5% RH − 9 Months | | | | | |
| Highest | 0.011 | 0.010 | 0.010 | 0.010 | 0.011 | White to |
| Unknown | | | | | | Off-White |
| Impurity (%) | | | | | | Suspension |
| Total | 0.027 | 0.027 | 0.027 | 0.030 | 0.025 | |
| Impurities | | | | | | |
| (%) | | | | | | |
| Assay (% of | 101.1 | 100.4 | 100.7 | 100.9 | 100.7 | |
| 6.66 mg/ml) | | | | | | |
| | 30 ± 2° C./75 ± 5% RH − 6 Months | | | | | |
| Highest | 0.008 | 0.007 | 0.007 | 0.007 | 0.007 | White to |
| Unknown | | | | | | Off-White |
| Impurity (%) | | | | | | Suspension |
| Total | 0.019 | 0.013 | 0.012 | 0.014 | 0.013 | |
| Impurities | | | | | | |
| (%) | | | | | | |
| Assay (% of | 99.7 | 99.7 | 100.0 | 99.8 | 100.0 | |
| 6.66 mg/ml) | | | | | | |

Mean Particle Size Parameters: D₁₀ = 3.00 μm, D₅₀ = 10.9 μm, D₉₀ = 31.5 μm

### Formulation 12C

| | 0 Hrs | | | | | |
|---|---|---|---|---|---|---|
| Time | (Initial) | 6 Hrs | 12 Hrs | 18 Hrs | 24 Hrs | Comments |
| | 25 ± 2° C./60 ± 5% RH − 9 Months | | | | | |
| Highest | 0.007 | 0.007 | 0.006 | 0.014 | 0.006 | White to |
| Unknown | | | | | | Off-White |
| Impurity (%) | | | | | | Suspension |
| Total | 0.021 | 0.013 | 0.011 | 0.028 | 0.016 | |
| Impurities | | | | | | |
| (%) | | | | | | |
| Assay (% of | 99.6 | 100.0 | 100.6 | 100.1 | 100.3 | |
| 6.66 mg/ml) | | | | | | |
| | 30 ± 2° C./65 ± 5% RH − 9 Months | | | | | |
| Highest | 0.012 | 0.014 | 0.011 | 0.008 | 0.011 | White to |
| Unknown | | | | | | Off-White |
| Impurity (%) | | | | | | Suspension |
| Total | 0.030 | 0.036 | 0.024 | 0.015 | 0.019 | |
| Impurities | | | | | | |
| (%) | | | | | | |
| Assay (% of | 100.6 | 100.3 | 99.5 | 100.3 | 100.5 | |
| 6.66 mg/ml) | | | | | | |
| | 30 ± 2° C./75 ± 5% RH − 6 Months | | | | | |
| Highest | 0.008 | 0.006 | 0.009 | 0.006 | 0.009 | White to |
| Unknown | | | | | | Off-White |
| Impurity (%) | | | | | | Suspension |
| Total | 0.014 | 0.017 | 0.015 | 0.017 | 0.016 | |
| Impurities | | | | | | |
| (%) | | | | | | |
| Assay (% of | 99.6 | 99.6 | 100.0 | 99.9 | 99.9 | |
| 6.66 mg/ml) | | | | | | |

Mean Particle Size Parameters: D₁₀ = 2.91 μm, D₅₀ = 12.2 μm, D₉₀ = 44.3 μm.

All references and patent publications cited herein are hereby incorporated by reference.

1. An oral pharmaceutical composition comprising a compound of Formula I or a pharmaceutically acceptable salt thereof: 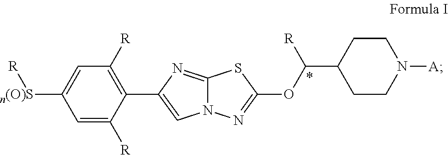
wherein
each R is independently selected from H, halogen, substituted or unsubstituted C₁₋₄ alkyl, and substituted or unsubstituted C₁₋₄ alkoxy;
A is selected from H, substituted or unsubstituted C₁₋₄ alkyl, substituted or unsubstituted phenyl, substituted or unsubstituted (C₁₋₄ alkyl)phenyl, substituted or unsubstituted 5-6 membered heteroaryl containing one or more heteroatoms selected from N, O, and S, substituted or unsubstituted heterocyclyl containing one or more heteroatom selected from N, O, and S, and substituted or unsubstituted C₁₋₄ alkoxycarbonyl; wherein the substituents are selected from halogen, substituted or unsubstituted C₁₋₄ alkyl, and substituted or unsubstituted C₁₋₄ alkoxy;
n is 0, 1, 2, or 3; and
* denotes a stereocenter;
and one or more pharmaceutically acceptable excipients,
wherein the compound of Formula I has a particle size D₉₀ of less than about 200 μm.

2. The oral pharmaceutical composition of claim 1, wherein the compound of Formula I is a compound of Formula IA, IB, IC, ID or IE:

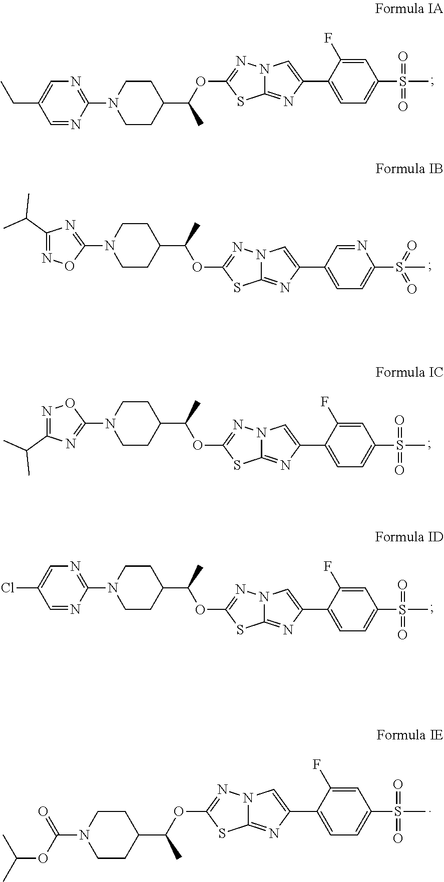

3. The oral pharmaceutical composition of claim 1, wherein the compound of Formula I is a compound of Formula IA:

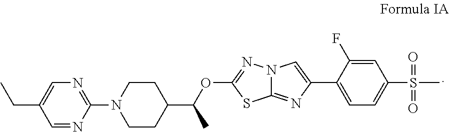 [SMILES: CCc1cnc(N2CCC([C@H](C)Oc3nn4cc(-c5ccc(S(C)(=O)=O)cc5F)nc4s3)CC2)nc1]

4. The oral pharmaceutical composition of claim 1, wherein the compound of Formula I has a particle size D₉₀ of between about 60 μm and about 100 μm.

5. The oral pharmaceutical composition of claim 1, wherein the compound of Formula I has a particle size Doo of between about 2.0 μm and about 150 μm.

6. The oral pharmaceutical composition of claim 1, wherein the compound of Formula I represents less than about 75% w/w of the total weight of the composition.

7. The oral pharmaceutical composition of claim 1, wherein the composition is a tablet or a capsule.

8. The oral pharmaceutical composition of claim 1, wherein the composition is a solid, and at least 90% of the compound of Formula I in the composition is released within 30 minutes when tested according to USP II (paddle method) at 100 rpm and 37.0±0.5° C. in 900 mL of (i) simulated gastric fluid (without pepsin) at pH 1.2, (ii) 0.01 N HCl aqueous solution containing 1% sodium lauryl sulfate, (iii) 0.1 N HCl aqueous solution containing 1% sodium lauryl sulfate, (iv) aqueous pH 4.5 acetate solution and 1% sodium lauryl sulfate, or (v) aqueous pH 6.8 phosphate solution and 1% sodium lauryl sulfate.

9. The oral pharmaceutical composition of claim 1, wherein the composition is a solution or a suspension.

10. A compressed tablet comprising a compound of Formula IA or a pharmaceutically acceptable salt thereof 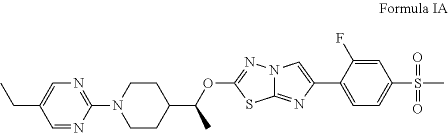 [SMILES: CCc1cnc(N2CCC([C@H](C)Oc3nn4cc(-c5ccc(S(C)(=O)=O)cc5F)nc4s3)CC2)nc1]
dispersed in
(a) a non-ionic, water dispersible surfactant;
(b) colloidal silicon dioxide;
(c) crospovidone; and
(d) spray-dried (i) D-mannitol, (ii) xylitol, (iii) microcrystalline cellulose, (iv) crospovidone, and (v) dibasic calcium phosphate;
wherein the compound of Formula IA has a particle size D₉₀ of less than about 200 μm.

11. The tablet of claim 10, wherein the non-ionic, water dispersible surfactant is a mixture of lauroyl polyoxyl-32 glycerides and PEG 6000.

12. An oral aqueous suspension comprising:
(a) a compound of Formula IA or a pharmaceutically acceptable salt thereof 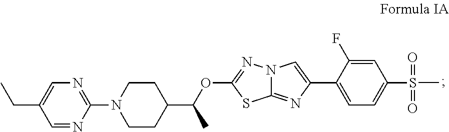 [SMILES: CCc1cnc(N2CCC([C@H](C)Oc3nn4cc(-c5ccc(S(C)(=O)=O)cc5F)nc4s3)CC2)nc1]
and
(b) 2-hydroxypropyl beta-cyclodextrin.

13. The oral aqueous suspension of claim 12, wherein the ratio of the compound of Formula IA to 2-hydroxypropyl beta-cyclodextrin is about 1:5 to about 1:10.

14. A method of treating diabetes or obesity comprising orally administering to a patient in need thereof an oral pharmaceutical composition according to claim 1.
